SEQUENCE LISTING (29 sequences)

SEQ ID NO: 1 - protein, 389 aa, Artificial Sequence
  Synthetic Proteins
MMASMTGGQQMGHHHHHHHDDDDKLDYAVTGRGDSPASSKPIAVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPLDYAVTGRGDSPASSKPIAVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPLDYAVTGRGDSPASSKPIAVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPLE

SEQ ID NO: 2 - protein, 5 aa, Artificial Sequence
  Synthetic Proteins
VPGVG

SEQ ID NO: 3 - protein, 5 aa, Artificial Sequence
  Synthetic Proteins
VPGKG

SEQ ID NO: 4 - protein, 5 aa, Artificial Sequence
  Synthetic Proteins
VPGIG

SEQ ID NO: 5 - protein, 59 aa, ARTIFICIAL SEQUENCE
  Synthetic Proteins
GAGAGSGAGAGSGAGAGSGAGAGSGAGAGSGAGAGSGAGAGSGAGAGSGAGAGSGAAGY

SEQ ID NO: 6 - protein, 6 aa, Artificial Sequence
  Synthetic Proteins
GAGAGS

SEQ ID NO: 7 - protein, 71 aa, Artificial Sequence
  Synthetic Proteins
GAGAGSGAGAGSGAGAGSGAGAGSGAGAGSGAGAGSGAGAGSGAGAGSGAGAGSGAAVTGRGDSPASAAGY

SEQ ID NO: 8 - protein, 74 aa, Artificial Sequence
  Synthetic Proteins
GAGAGSGAGAGSGAGAGSGAGAGSGAGAGSGAGAGSGAGAGSGAGAGSGAGAGSGAAPGASIKVAVSAGPSAGY

SEQ ID NO: 9 - protein, 73 aa, Artificial Sequence
  Synthetic Proteins
GAGAGSGAGAGSGAGAGSGAGAGSGAGAGSGAGAGSGAGAGSGAGAGSGAGAGSGAAPGASIKVAVSGPSAGY

SEQ ID NO: 10 - protein, 71 aa, Artificial Sequence
  Synthetic Proteins
GAGAGSGAGAGSGAGAGSGAGAGSGAGAGSGAGAGSGAGAGSGAGAGSGAGAGSRYVVLPRPVCFEKAAGY

SEQ ID NO: 11 - protein, 20 aa, Artificial Sequence
  Synthetic Proteins
VPGVGVPGVGVPGVGVPGVG

SEQ ID NO: 12 - protein, 52 aa, Artificial Sequence
  Synthetic Proteins
GVGVPGVGVPGVGVPGVGVPGVGVPGVGVPGVGVPGVGVPGAGAGSGAGAGS

SEQ ID NO: 13 - protein, 82 aa, Artificial Sequence
  Synthetic Proteins
GAAVPGVGVPGVGVPGVGVPGVGVAAGYGAGAGSGAGAGSGAGAGSGAGAGSGAGAGSGAGAGSGAGAGSGAGAGSGAGAGS

SEQ ID NO: 14 - protein, 129 aa, Artificial Sequence
  Synthetic Proteins
GAGAGSGAGAGSGAGAGSGAGAGSGAGAGSGAGAGSGAAGYGAGAGSGAGAGSGAGAGSGAGAGSGAGAGSGAGAGSGAGAGSGAGAGSGVGVPGVGVPGVGVPGVGVPGVGVPGVGVPGVGVPGVGVP

SEQ ID NO: 15 - protein, 88 aa, Artificial Sequence
  Synthetic Proteins
GVGVPGVGVPGVGVPGVGVPGVGVPGVGVPGVGVPGVGVPGAGAGSGAGAGSGAGAGSGAGAGSGAGAGSGAGAGSGAGAGSGAGAGS

SEQ ID NO: 16 - protein, 108 aa, Artificial Sequence
  Synthetic Proteins
GVGVPGVGVPGVGVPGVGVPGVGVPGVGVPGVGVPGVGVPGVGVPGVGVPGVGVPGVGVPGAGAGSGAGAGSGAGAGSGAGAGSGAGAGSGAGAGSGAGAGSGAGAGS

SEQ ID NO: 17 - protein, 128 aa, Artificial Sequence
  Synthetic Proteins
GVGVPGVGVPGVGVPGVGVPGVGVPGVGVPGVGVPGVGVPGVGVPGVGVPGVGVPGVGVPGVGVPGVGVPGVGVPGVGVPGAGAGSGAGAGSGAGAGSGAGAGSGAGAGSGAGAGSGAGAGSGAGAGS

SEQ ID NO: 18 - protein, 208 aa, Artificial Sequence
  Synthetic Proteins
GVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGAGAGSGAGAGSGAGAGSGAGAGSGAGAGSGAGAGSGAGAGSGAGAGS

SEQ ID NO: 19 - protein, 76 aa, Artificial Sequence
  Synthetic Proteins
GVGVPGVGVPGVGVPGVGVPGVGVPGVGVPGVGVPGVGVPGAGAGSGAGAGSGAGAGSGAGAGSGAGAGSGAGAGS

SEQ ID NO: 20 - protein, 64 aa, Artificial Sequence
  Synthetic Proteins
GVGVPGVGVPGVGVPGVGVPGVGVPGVGVPGVGVPGVGVPGAGAGSGAGAGSGAGAGSGAGAGS

SEQ ID NO: 21 - protein, 56 aa, Artificial Sequence
  Synthetic Proteins
AKLKLAEAKLELAEAKLKLAEAKLELAEAKLKLAEAKLELAEAKLKLAEAKLELAE

SEQ ID NO: 22 - protein, 15 aa, Artificial Sequence
  Synthetic Proteins
GAPGPPGPPGPPGPP

SEQ ID NO: 23 - protein, 39 aa, Artificial Sequence
  Synthetic Proteins
GAPGPPGPPGPPGPPGAPGPPGPPGPPGPPGPAGPVGSP

SEQ ID NO: 24 - protein, 63 aa, Artificial Sequence
  Synthetic Proteins
GAPGPPGPPGPPGPPGAPGPPGPPGPPGPPGLPGPKGDRGDAGPKGADGSPGPAGPAGPVGSP

SEQ ID NO: 25 - protein, 15 aa, Artificial Sequence
  Synthetic Proteins
GAPGAPGSQGAPGLQ

SEQ ID NO: 26 - protein, 17 aa, Artificial Sequence
  Synthetic Proteins
YAVTGRGDSPASSKPIA

SEQ ID NO: 27 - protein, 22 aa, Artificial Sequence
  Synthetic Proteins
LEGEEIQIGHIPREDVDYHLYP

SEQ ID NO: 28 - protein, 2355 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 29 - protein, 757 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

(19) United States
(12) Patent Application Publication
(10) Pub. No.: US 2010/0261652 A1
(43) Pub. Date: Oct. 14, 2010

(54) Tissue Adhesive Using Engineered Proteins

(75) Inventors: Muzhou Wang, Cincinnati, OH (US); Matthew S. Mattson, Pasadena, CA (US); David A. Tirrell, Pasadena, CA (US); Julia A. Kornfield, Pasadena, CA (US)

Correspondence Address: Karin E. Peterka, Attorney at Law, 610 Brown Court, Altadena, CA, 91001, US

(73) Assignee: California Institute of Technology, Pasadena, CA (US)

(21) Appl. No.: 12/749,507

(22) Filed: Mar. 29, 2010

Related U.S. Application Data
(60) Provisional application No. 61/167,731, filed on Apr. 8, 2009.

(51) Int. Cl. A61K 38/16; A61K 38/08; A61K 38/10; A61P 27/02; A61F 2/14

(52) U.S. Cl. 514/12; 514/17; 514/13; 623/5.11

(57) ABSTRACT

There is provided in one embodiment of the disclosure a tissue adhesive composition comprising an engineered protein having repeated blocks of an elastin domain and at least one cell-binding domain and further comprising a polymer crosslinker. When the engineered protein and the polymer crosslinker are introduced onto a tissue, the engineered protein and the polymer crosslinker initiate an in situ crosslinking reaction to form an adhesive bond that is mechanically strong, transparent, biocompatible, and stimulates regrowth of one or more tissue layers over the adhesive bond. In another embodiment of the disclosure there is provided a molded corneal onlay and method of making the same.

### CROSS-REFERENCE TO RELATED APPLICATION

This application claims priority of U.S. Provisional Patent Application Ser. No. 61/167,731, filed Apr. 8, 2009, which is incorporated herein by reference in its entirety.

### BACKGROUND

a. Field

The disclosure relates to tissue adhesives using engineered proteins. In particular, the disclosure relates to tissue adhesives using engineered proteins for use in biomedical applications such as opththalmic repair and moldings.

b. Background Art

The problem of adhering two tissue surfaces has been important since the beginning of medicine. In opthalmology, tissue adhesion is widely used for corneal repair necessitated by surgery, injury, or disease. It has been estimated that 1 million patients per year seek treatment for corneal wounds in the United States (May et al., “The Epidemiology of Serious Eye Injuries from the United States Eye Injury Registry”, Graefe's Archive for Clinical and Experimental Opthalmology, 238:153-157 (2000)). Additionally, tissue adhesion is necessary for permanently implanting therapeutic materials.

In the field of tissue adhesion, sutures have been and still are the gold standard (Lauto et al., “Adhesive Biomaterials for Tissue Reconstruction”, Journal of Chemical Technology and Biotechnology, 83:464-472 (2008)). While sutures are commonly used because they have excellent mechanical bond strength, they have disadvantages over other adhesion methods. Sutures are inherently invasive and do not actively participate in tissue healing (Grinstaff M. W., “Designing Hydrogel Adhesives for Corneal Wound Repair”, Biomaterials, 28:5205-5214 (2007)). Insertion of the sutures can cause extraneous trauma to sensitive tissues. Sutures can provide avenues of infection and loci for scarring. Suturing is an advanced technical skill, and the efficacy of the sutures depends on the surgeon who placed them.

Recently, tissue adhesives have emerged as a promising alternative to sutures in the cornea, either completely replacing sutures or working in tandem (Lauto et al., “Adhesive Biomaterials for Tissue Reconstruction”, Journal of Chemical Technology and Biotechnology, 83:464-472 (2008)). Over 2 million annual surgical procedures in the United States could benefit from the use of a corneal adhesive (Steinberg et al., “The Content and Cost of Cataract Surgery”, Archives of Opthalmology, 111:1041-1049 (1993)). These surgeries include LASIK (laser-assisted in situ keratomileusis), keratoplasty, and cataract removal. However, use of these adhesives can be challenging since the cornea must retain its shape and optical clarity in order to maintain proper function. Many common tissue adhesives in the literature are attractive for this application, but each has its own shortcomings. For example, cyanoacrylates provide excellent bond strength, but cytotoxicity can be a major issue, especially since the small molecule degradation products diffuse quickly into surrounding tissues (Vote et al., “Cyanoacrylate Glue for Corneal Perforations: A Description of a Surgical Technique and a Review of the Literature”, Clinical and Experimental Opthalmology, 28:437-442 (2000)). Fibrin glues avoid the cytotoxicity issue, however, viral transmission can be problematic since the components are of animal origin, and preparation and application can be complex (Lagoutte et al., “A Fibrin Sealant for Perforated and Preperforated Corneal Ulcers”, British Journal of Opthalmology, 73:757-761 (1989); Kim et al., “Tissue Adhesives in Corneal Cataract Incisions”, Current Opinion in Opthalmology, 18:39-43 (2007)).

A parallel problem in corneal wound repair is epithelial cell adhesion. The corneal epithelium is a thin protective layer of cells that covers the cornea. It provides transparency to the stroma of the eye, a barrier against fluid loss, and a first line of defense against microbial infection (Oyster C. W., The Human Eye: Structure and Function, Chapter 8, 332-335, Sinauer Associates, Inc., Sunderland, Mass. (1999)). Since the epithelium is easily destroyed in corneal trauma, successful therapies for corneal wounds must facilitate epithelial regeneration (Klenkler et al., “EGF-grafted PDMS Surfaces in Artificial Cornea Applications”, Biomaterials, 26:7286-7296 (2005)). In the case of tissue adhesives, foreign materials introduced in the cured adhesive bond must have favorable cell adhesive properties that allow the epithelium to regrow over it.

Known materials with cell adhesive properties have been demonstrated in the literature (Liu et al., “Comparative Cell Response to Artificial Extracellular Matrix Proteins Containing the RGD and CS5 Cell-binding Domains”, Biomacromolecules, 5:497-504 (2004); Rizzi et al., “Recombinant Protein-co-PEG Networks as Cell-adhesive and Proteolytically Degradable Hydrogel Matrixes. Part I: Development and Physicochemical Characteristics”, Biomacromolecules, 6:1226-1238 (2005); Heilshorn et al., “Endothelial Cell Adhesion to the Fibronectin CS5 Domain in Artificial Extracellular Matrix Proteins”, Biomaterials, 24:4245-4252 (2003); Duan et al., “Biofunctionalization of Collagen for Improved Biological Response: Scaffolds for Corneal Tissue Engineering”, Biomaterials, 28:78-88 (2007)). In particular, Tirrell et al. have successfully engineered an artificial protein (known as aECM) with the desired ability to promote epithelial regrowth (U.S. Pat. No. 7,229,634 to Tirrell et al., entitled “Engineered Proteins, and Methods of Making and Using”). Known art (U.S. Pat. No. 7,229,634) teaches the use of this artificial protein aECM in the form of crosslinked films as corneal onlays. These films have shown favorable ophthalmic properties such as transparency and biocompatibility.

However, a proper means of physically attaching these onlays to the stroma of the eye is still lacking, and thus actual application of these onlays is currently difficult (Nowatzki P. J., “Characterization of Crosslinked Artificial Protein Films”, Doctoral Thesis, California Institute of Technology, Pasadena, Calif., 2006). In the study disclosed by Nowatzki, a liquid aECM solution was formed into a solid corneal onlay using a bifunctional sulfosuccinimidyl-ester (BS3) to crosslink primary amines present in the aECM sequence. These onlays were then implanted in vivo into a stromal pocket of a rabbit cornea (see FIGS. 10A-10C discussed below).

The characteristics of a preferred tissue adhesive, such as a corneal adhesive, include: (1) mechanical strength, (2) transparency, (3) facile application, (4) biocompatibility, and (5) rapid epithelium regrowth over the wound site or adhesive bond. Known tissue adhesives do not satisfy all of these criteria. There is currently an unmet need for a formulation that meets all of these criteria for a preferred corneal adhesive. In addition, there is an unmet need for a tissue adhesive formulation, such as a corneal adhesive formulation, that secures a corneal onlay comprising an engineered protein, such as aECM, to the corneal stroma.

### SUMMARY

This need for a tissue adhesive, such as a corneal adhesive, having the following characteristics: (1) mechanical strength, (2) transparency, (3) facile application, (4) biocompatibility, and (5) rapid epithelium regrowth over the wound site or adhesive bond, is met in this disclosure. In addition, this need for a tissue adhesive formulation, such as a corneal adhesive formulation, that secures a corneal onlay comprising an engineered protein, such as aECM, to the corneal stroma, is met in this disclosure.

In one embodiment of the disclosure, there is provided a tissue adhesive composition comprising an engineered protein having repeated blocks of an elastin domain and at least one cell-binding domain, and further comprising a polymer crosslinker. When the engineered protein and the polymer crosslinker are introduced onto a tissue, the engineered protein and the polymer crosslinker initiate an in situ crosslinking reaction to form an adhesive bond that is mechanically strong, transparent, biocompatible, and stimulates regrowth of one or more tissue layers over the adhesive bond. Preferably, the tissue adhesive composition comprises about 10% weight per volume (w/v) to about 40% weight per volume (w/v) of an engineered protein based on the total weight per volume of the tissue adhesive composition. Preferably, the engineered protein comprises aECM-RGD comprising SEQ ID NO: 1. Preferably, the tissue adhesive composition further comprises about 10% weight per volume (w/v) to about 40% weight per volume (w/v) of a polymer crosslinker based on the total weight per volume of the tissue adhesive composition. Preferably, the polymer crosslinker may comprise a linear telechelic PEG (polyethylene glycol), a star PEG with two or more arms, or another suitable polymer crosslinker. More preferably, the polymer crosslinker may comprise a four-arm polyethylene glycol with succinimidyl glutarate end groups (PEG-S). Preferably, the tissue adhesive composition comprises a corneal adhesive for use in a mammalian eye. When the engineered protein and the polymer crosslinker are introduced onto a tissue, the engineered protein and the polymer crosslinker initiate an in situ crosslinking reaction to form an adhesive bond that is mechanically strong, optically transparent, biocompatible, and stimulates regrowth of one or more tissue layers over the adhesive bond.

In one embodiment, there is provided a method to provide a tissue adhesive composition. The method comprises combining an engineered protein and a polymer crosslinker, the engineered protein comprising repeated blocks of an elastin domain and at least one cell-binding domain. Preferably, the combining is performed by providing engineered protein in about 10% weight per volume (w/v) to about 40% weight per volume (w/v) based on the total weight per volume of the resulting tissue adhesive composition and combining said engineered protein with the polymer cross-linker. Preferably, the engineered protein comprises aECM-RGD comprising SEQ ID NO: 1. Preferably, the combining is performed by providing about 10% weight per volume (w/v) to about 40% weight per volume (w/v) of a polymer crosslinker based on the total weight per volume of the resulting tissue adhesive composition and combining said polymer cross-linker with the engineered protein, possibly also provided in the above mentioned range. Preferably, the polymer crosslinker may comprise a linear telechelic PEG (polyethylene glycol), a star PEG with two or more arms, or another suitable polymer crosslinker. More preferably, the polymer crosslinker may comprise a four-arm polyethylene glycol with succinimidyl glutarate end groups (PEG-S).

In one embodiment, there is provided a system to provide a tissue adhesive composition. The system comprises an engineered protein and a polymer crosslinker, the engineered protein comprising repeated blocks of an elastin domain and at least one cell-binding domain. Preferably, the polymer crosslinker may comprise a linear telechelic PEG (polyethylene glycol), a star PEG with two or more arms, or another suitable polymer crosslinker. More preferably, the polymer crosslinker may comprise a four-arm polyethylene glycol with succinimidyl glutarate end groups (PEG-S). Preferably, the tissue adhesive composition comprises a corneal adhesive for use in a mammalian eye. When the engineered protein and the polymer crosslinker are introduced onto a tissue, the engineered protein and the polymer crosslinker initiate an in situ crosslinking reaction to form an adhesive bond that is mechanically strong, optically transparent, biocompatible, and stimulates regrowth of one or more tissue layers over the adhesive bond.

In another embodiment of the disclosure, there is provided a molded corneal onlay for use in a mammalian eye, which can include use in treatment of a cornea, such as use in treatment of a corneal implant. The molded corneal onlay comprises a bulk hydrogel comprising an engineered protein having repeated blocks of an elastin domain and at least one cell-binding domain, and further comprising a polymer crosslinker. The bulk hydrogel is preferably molded on a corneal surface to form a molded corneal onlay, and the engineered protein and the polymer crosslinker initiate an in situ crosslinking reaction to attach the molded corneal onlay to the corneal surface. In one embodiment, the molded corneal onlay can be molded in vitro, e.g., on a corneal tissue or on an artificial surface mimicking a corneal surface, before application on the corneal surface in a human or animal body. In one embodiment, the molded corneal onlay can be molded in vivo, e.g., directly on the corneal surface of the individual where the molded corneal onlay is finally applied. The molded corneal onlay is optically transparent, biocompatible, protects the corneal surface, is used to correct refractive errors, and stimulates cellular regrowth of corneal cells.

In another embodiment of the disclosure, there is provided a method of adhering tissue in in vitro and/or in vivo applications. The method comprises applying a tissue adhesive composition to one or more tissue surfaces. The tissue adhesive composition comprises an engineered protein having repeated blocks of an elastin domain and at least one cell-binding domain, and further comprises a polymer crosslinker. When the engineered protein and the polymer crosslinker are applied to the one or more tissue surfaces, the engineered protein and the polymer crosslinker initiate an in situ crosslinking reaction to form an adhesive bond. In one embodiment, the engineered protein and polymer crosslinker are applied to the one or more tissue surfaces in vitro, possibly before applying the tissue surfaces comprising the engineered protein and polymer crosslinker on a human or animal body. In one embodiment, the engineered protein and polymer crosslinker are applied to the one or more tissue surfaces in vivo, e.g., directly on a human or animal tissue such as cornea. The method further comprises curing the tissue adhesive composition to bond the composition to the one or more tissue surfaces and to provide a cured adhesive bond that is mechanically strong, transparent, biocompatible, and stimulates regrowth of one or more tissue layers over the cured adhesive bond. In one embodiment the curing is performed in vitro possibly before applying the cured tissue surface on the human or animal body. In one embodiment, the curing is performed on a tissue in vivo. Preferably, the tissue adhesive composition comprises about 10% weight per volume (w/v) to about 40% weight per volume (w/v) of an engineered protein based on the total weight per volume of the tissue adhesive composition. Preferably, the engineered protein comprises aECM-RGD comprising SEQ ID NO: 1. Preferably, the tissue adhesive composition further comprises about 10% weight per volume (w/v) to about 40% weight per volume (w/v) of a polymer crosslinker based on the total weight per volume of the tissue adhesive composition. Preferably, the polymer crosslinker may comprise a linear telechelic PEG (polyethylene glycol), a star PEG with two or more arms, or another suitable polymer crosslinker. More preferably, the polymer crosslinker may comprise a four-arm polyethylene glycol with succinimidyl glutarate end groups (PEG-S).

In one embodiment of the disclosure, there is provided a system for adhering a tissue. The system comprises a tissue adhesive composition and at least one tissue substrate for simultaneous sequential or separate use in a method of adhering tissue herein described. In one embodiment, the system comprises two or more tissue substrates and at least one of the applying and the curing is performed in vitro.

In another embodiment of the disclosure, there is provided a method of making a molded corneal onlay for use in a mammalian eye. The method comprises providing a bulk hydrogel comprising an engineered protein having repeated blocks of an elastin domain and at least one cell-binding domain, and further comprising a polymer crosslinker. The method further comprises molding the bulk hydrogel on a corneal surface in vitro or in vivo to form a molded corneal onlay. In one embodiment, the molded corneal onlay can be molded in vitro, e.g., on a corneal tissue substrate or on an artificial surface mimicking a corneal surface, before application on the corneal surface in a human or animal body. In one embodiment, the molded corneal onlay can be molded in vivo, e.g., directly on the corneal surface of the individual where the molded corneal onlay is finally applied. In one embodiment, the method can further comprise attaching the molded corneal onlay to the corneal surface via the engineered protein and the polymer crosslinker initiating an in situ crosslinking reaction. In particular, in one embodiment, attaching can be performed in vitro, e.g., on a corneal surface outside a human or animal body. In one embodiment, the attaching can be performed in vivo and in particular on a corneal surface of an individual to which the molded corneal onlay is attached. The molded corneal onlay formed is optically transparent, biocompatible, protects the corneal surface, is used to correct refractive errors, and stimulates cellular regrowth of corneal cells.

In one embodiment of the disclosure, there is provided a system for providing a molded corneal onlay for use in a mammalian eye. The system comprises a bulk hydrogel and a corneal tissue substrate for simultaneous sequential or separate use in a method of making a molded corneal onlay herein described wherein at least one of the molding and the attaching is performed in vitro.

The features, functions, and advantages that have been discussed can be achieved independently in various embodiments of the disclosure or may be combined in yet other embodiments further details of which can be seen with reference to the following description and drawings.

### BRIEF DESCRIPTION OF THE DRAWINGS

The disclosure can be better understood with reference to the following detailed description taken in conjunction with the accompanying drawings which illustrate preferred and exemplary embodiments, but which are not necessarily drawn to scale, wherein:

FIG. 1 is an illustration of the protein design for the engineered protein material aECM-RGD;

FIG. 2 is an illustration of a chemical structure of PEG-S polymer crosslinker;

FIG. 3 is an illustration of a front view in partial cross-section of a shear rheometer;

FIG. 4A is a schematic illustration of a mock surgery showing PEG-S/aECM adhesive applied to a gelatin disk;

FIG. 4B is a schematic illustration of a mock surgery showing the gelatin disk of FIG. 4A applied to the stroma of an eye to form a corneal onlay;

FIG. 5A is a schematic illustration of a mock surgery showing a cut anterior portion of the stroma of an eye;

FIG. 5B is a schematic illustration of a mock surgery showing the cut anterior portion of FIG. 5A removed and a PEG-S/aECM adhesive applied to a cavity opening in the stroma;

FIG. 5C is a schematic illustration of a mock surgery showing the removed cut anterior portion of FIG. 5B reattached to the stroma with PEG-S/aECM adhesive;

FIG. 6A is a schematic illustration of a mock surgery showing a glass lens over the stroma of an eye;

FIG. 6B is a schematic illustration of a mock surgery showing a PEGS/aECM adhesive inserted between the glass lens and the stroma of the eye;

FIG. 6C is a schematic illustration of a mock surgery showing a molded in situ corneal onlay on the stroma of an eye;

FIG. 7 is an illustration of a bar graph showing shear stress at failure compared between different interfacial conditions;

FIG. 8 is an illustration of a graph showing PEG-S/aECM gelation time versus pH of PCx buffer;

FIG. 9 is an illustration of a graph showing the direct transmittance versus wavelength of cured PEG-S/aECM adhesive compared to an uncured PEG-S/aECM adhesive;

FIG. 10A shows a clinical exam photograph depicting results from an in vivo study of epithelium regrowth over an aECM-RGD film on a rabbit cornea immediately after implantation;

FIG. 10B shows a clinical exam photograph depicting results from an in vivo study of epithelium regrowth over an aECM-RGD film on a rabbit cornea two days after implantation;

FIG. 10C shows a clinical exam photograph depicting results from an in vivo study of epithelium regrowth over an aECM-RGD film on a rabbit cornea seven days after implantation;

FIG. 11A shows a clinical exam photograph of a mock surgery performed on a porcine cadaver eye depicting a gelatin disk adhered to the stroma of the eye with PEG-S/aECM adhesive;

FIG. 11B shows a clinical exam photograph of a mock surgery performed on a porcine cadaver eye depicting forceps applying shear force to the gelatin disk of FIG. 11A;

FIG. 12A shows a clinical exam photograph of a mock surgery performed on a porcine cadaver eye depicting a side view of a reattached piece of stroma of the eye reattached with PEG-S/aECM adhesive;

FIG. 12B shows a clinical exam photograph of a mock surgery performed on a porcine cadaver eye depicting a cavity opening in the stroma of the eye where the reattached piece of stroma of FIG. 12A was removed along with a piece of surrounding stroma;

FIG. 12C shows a clinical exam photograph of a mock surgery performed on a porcine cadaver eye depicting the removed reattached piece of stroma with the piece of surrounding stroma still attached via the PEG-S/aECM adhesive;

FIG. 13A shows a clinical exam photograph of a mock surgery performed on a porcine cadaver eye depicting a molded in situ corneal onlay molded between a glass lens and the stroma of the eye;

FIG. 13B shows a clinical exam photograph of a mock surgery performed on a porcine cadaver eye depicting a top view of the molded in situ corneal onlay of FIG. 13A; and,

FIG. 13C shows a clinical exam photograph of a mock surgery performed on a porcine cadaver eye depicting a side view of the molded in situ corneal onlay of FIG. 13A.

### DETAILED DESCRIPTION

Disclosed embodiments will now be described more fully hereinafter with reference to the accompanying drawings, in which some, but not all disclosed embodiments are shown. Indeed, several different embodiments may be provided and should not be construed as limited to the embodiments set forth herein. Rather, these embodiments are provided so that this disclosure will be thorough and complete and will fully convey the scope of the disclosure to those skilled in the art.

In one embodiment of the disclosure, there is provided a tissue adhesive, such as a corneal adhesive, having the following characteristics: (1) mechanical strength, (2) transparency, (3) facile application, (4) biocompatibility, and (5) rapid epithelium regrowth over the wound site or adhesive bond. In another embodiment of the disclosure, there is provided a tissue adhesive formulation, such as a corneal adhesive formulation, that secures a corneal onlay comprising an engineered protein, such as aECM, to the corneal stroma. The essence of the disclosure is captured by the in situ polymerization of aECM and a polymer crosslinker for use as a tissue adhesive, such as a corneal adhesive.

Embodiments of the corneal adhesive disclosed herein preferably include engineered proteins, such as those disclosed in U.S. Pat. No. 7,229,634 to Tirrell et al., entitled “Engineered Proteins, and Methods of Making and Using”, which is incorporated herein by reference in its entirety. More preferably, embodiments of the corneal adhesive disclosed herein include the engineered protein aECM, as disclosed in U.S. Pat. No. 7,229,634 to Tirrell, et al., in order to maximize cell adhesion to promote epithelium regrowth, including rapid or fast epithelium regrowth.

As used herein, the term “engineered protein” refers to a non-naturally-occurring polypeptide. The term encompasses, for example, a polypeptide that comprises one or more changes, including additions, deletions or substitutions, relative to a naturally occurring polypeptide, wherein such changes were introduced by recombinant DNA techniques. The term also encompasses a polypeptide that comprises an amino acid sequence generated by man, an artificial protein, a fusions protein, and a chimeric polypeptide. Those skilled in the art can readily generate engineered proteins useful according to this aspect of the disclosure. When several desired protein fragments or peptides are encoded in the nucleotide sequence incorporated into a vector, one of skill in the art will appreciate that the protein fragments or peptides may be separated by a spacer molecule such as, for example, a peptide, consisting of one or more amino acids. Generally, the spacer will have no specific biological activity other than to join the desired protein fragments or peptides together, or to preserve some minimum distance or other spatial relationship between them. However, the constituent amino acids of the spacer may be selected to influence some property of the molecule such as the folding, net charge, or hydrophobicity. Nucleotide sequences encoding for the production of residues which may be useful in purification of the expressed recombinant protein may be built into the vector. Such sequences are known in the art. For example, a nucleotide sequence encoding for a poly histidine sequence may be added to a vector to facilitate purification of the expressed recombinant protein on a nickel column. Once expressed, recombinant peptides, polypeptides, and proteins can be purified according to standard procedures known to one of ordinary skill in the art, including ammonium sulfate precipitation, affinity columns, column chromatography, gel electrophoresis, and the like. Substantially pure compositions of about 50% to about 99% homogeneity are preferred, and 80% to 95% or greater homogeneity are most preferred for use as therapeutic agents. Engineered proteins may be produced by any means, including, for example, peptide, polypeptide, or protein synthesis.

The terms “polypeptide”, “peptide”, or “protein” are used herein to designate a linear series of amino acid residues connected one to the other by peptide bonds between the alpha-amino and carboxy groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids. Furthermore, it should be noted that a dash at the beginning or end of an amino acid residue sequence indicates either a peptide bond to a further sequence of one or more amino acid residues or a covalent bond to a carboxyl or hydroxyl end group.

In one embodiment of the disclosure, the engineered protein aECM-RGD is used. The engineered protein aECM-RGD can provide, for example, mechanical strength and epithelium regrowth capabilities to the corneal adhesive. This artificial engineered protein can mimic key properties of the extracellular matrix. Its two functional domains are an elastin-like sequence that provides mechanical strength and flexibility, and a fibronectin-like cell-binding domain containing the RGD peptide sequence that promotes cell adhesion. Extensive studies have shown favorable mechanical and cell adhesion properties of this protein aECM within tissue engineering scaffolds and thin films for corneal onlays (DiZio et al., “Mechanical Properties of Artificial Protein Matrices Engineered for Control of Cell and Tissue Behavior”, Macromolecules, 36:1553-1558 (2003); Liu et al., “Comparative Cell Response to Artificial Extracellular Matrix Proteins Containing the RGD and CS5 Cell-binding Domains”, Biomacromolecules, 5:497-504 (2004); and, U.S. Pat. No. 7,229,634 to Tirrell et al., entitled “Engineered Proteins, and Methods of Making and Using”). In addition, coupling PEG with aECM does not inhibit cell binding (Liu et al., “Cell Response to RGD Density in Cross-Linked Artificial Extracellular Matrix Protein Films”, Biomacromolecules, 9:2984-2988 (2008)). Further, in vivo experiments on rabbit corneas have shown superior biocompatibility and epithelium regrowth over a crosslinked film of this protein (see Example 1 below). Although considerable research has been done on this protein, it is believed to be the first use of aECM-RGD as a component in a tissue adhesive, such as a corneal adhesive.

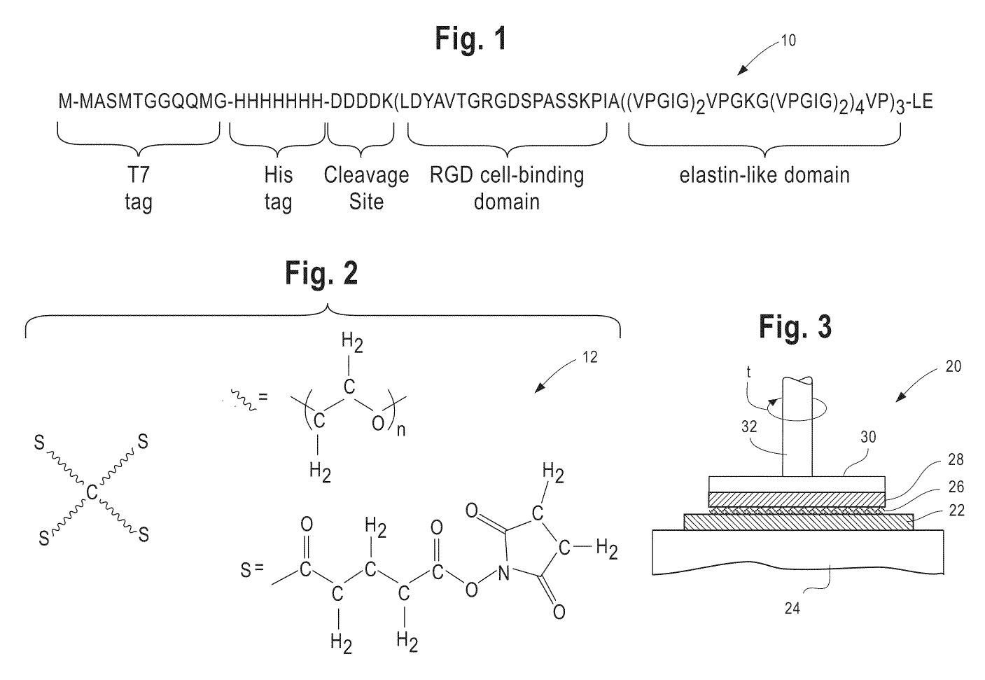

FIG. 1 is an illustration of a protein design 10 for engineered protein material aECM-RGD. The primary amino acid sequence for aECM-RGD is shown in FIG. 1. The engineered protein aECM-RGD is a hybrid, consisting of alternating domains from two natural sources. As used herein, a protein “domain” refers to a functional unit of a peptide sequence. For example, VPGIG is an elastin domain. For purposes of this disclosure, it is not necessary for a protein domain to have any particular structural or folding properties. As shown in FIG. 1, the engineered protein aECM-RGD includes an RGD cell-binding domain to promote interaction with the epithelial cells. A cell-binding domain with the RGD peptide sequence is derived from fibronectin and is known to support cell adhesion (Liu et al., “Comparative Cell Response to Artificial Extracellular Matrix Proteins Containing the RGD and CS5 Cell-binding Domains”, Biomacromolecules, 5:497-504 (2004)). As shown in FIG. 1, the engineered protein aECM-RGD further includes an elastin-like domain for structural support and flexibility. This second domain is derived from elastin and provides mechanical strength and flexibility (DiZio et al., “Mechanical Properties of Artificial Protein Matrices Engineered for Control of Cell and Tissue Behavior”, Macromolecules, 36:1553-1558 (2003)). While the native protein dissolves easily in water to form a liquid solution, preferably, the engineered protein material is in hydrogel form. Hence, multiple lysine residues may be engineered into the sequence to facilitate crosslinking by amine-reactive compounds. As shown in FIG. 1, the engineered protein aECM-RGD further includes a T7 tag at the N-terminus for protein or antibody identification, a 7 His tag for protein purification, and an enterokinase cleavage site for later removal of the epitope tags.

One example of a domain or protein fragment suitable for use in an engineered protein is an elastin domain. Elastin (SEQ ID NO: 29) is a structural molecule which offers great strength and flexibility. It is resistant to breakdown. Its sequence largely consists of simple repeating sequences of hydrophobic amino acids. An important feature of elastin which accounts for its unique structure and insolubility is its extensive crosslinking between polypeptide chains that occurs at lysine residues. One typical repeating sequence of elastin is VPGIG. Fibronectin (SEQ ID NO: 28) is a modular protein composed of homologous repeats of three prototypical types of domains known as types I, II, and III. Fibronectin type III (FN3) repeats are both the largest and the most common of the fibronectin subdomains. FN3 exhibits functional as well as structural modularity. Sites of interaction with other molecules have been mapped to short stretch of amino acids such as the Arg-Gly-Asp (RGD) sequence found in various FN3 domains. The RGD sequence is involved in interactions with integrin. Small peptides containing the RGD sequence can modulate a variety of cell adhesion invents associated with thrombosis, inflammation, and tumor metastasis. In the cornea, fibronectin is known to play an important role in wound healing. It can trigger epithelial cells to grow, migrate, and adhere to the underlying extracellular matrix. Some other proteins known to contain an FN3 domain include, but are not limited to, contactin 2 or axonin-1 protein, collagen alpha 1 chain, neural cell adhesion protein L1, leukocyte common antigen, and contactin protein.

The engineered proteins of the disclosure may further include any protein that, when crosslinked or formed into a shaped product, has favorable ophthalmic properties, such as elasticity, transparency, biocompatability, mechanical strength, facile application, epithelium regrowth, and the like. In certain embodiments of the disclosure, the engineered proteins are fusion proteins or chimeric proteins. In certain embodiments of the disclosure, the engineered proteins are made from a combination of human protein domains from various protein sources. In one embodiment of the disclosure, the engineered protein comprises at least one domain from a human extracellular matrix protein. In another embodiment of the disclosure, the human protein domain is a variant of the wild-type protein that has been modified to increase the favorable ophthalmic properties of the engineered protein.

Generally, blocks include groups of repeating amino acids making up a peptide sequence that occurs in a protein. Genetically engineered proteins are qualitatively distinguished from sequential polypeptides found in nature in that the length of their block repeats can be greater (up to several hundred amino acids versus less than ten for sequential polypeptides) and the sequence of their block repeats can be almost infinitely complex. Table 1 depicts examples of genetically engineered blocks. Table 1 and a further description of genetically engineered blocks may be found in Franco A. Ferrari and Joseph Cappello, Biosynthesis of Protein Polymers, in: Protein-Based Materials, (eds., McGrath et al.), Chapter 2, pp. 37-60, Birkhauser, Boston (1997). An engineered protein may comprise any of the below sequences in any order, in any number of repeats, and in combination with any other suitable domain, such as a fibronectin domain or an elastin domain, to provide a protein that, when formed into a lens or artificial tissue, provides favorable ophthalmic properties. The engineered proteins may also include functional variants of any of the sequences described herein. In certain embodiments, a functional variant has at least 80% sequence homology to its reference sequence.

TABLE 1

Protein polymer sequences

| | |
|---|---|
| Polymer Name | Monomer Amino Acid Sequence |

| | | |
|---|---|---|
| SLP 3 | (SEQ ID NO: 5) | [(GAGAGS)₉GAAGY)] |
| SLP 4 | (SEQ ID NO: 6) | (GAGAGS)ₙ |
| SLP F | (SEQ ID NO: 7) | [(GAGAGS)₉GAAVTGRGDSPASAAGY]ₙ |
| SLP L3.0 | (SEQ ID NO: 8) | [(GAGAGS)₉GAAPGASIKVAVSAGPSAGY]ₙ |
| SLP L3.1 | (SEQ ID NO: 9) | [(GAGAGS)₉GAAPGASIKVAVSGPSAGY]ₙ |
| SLP F9 | (SEQ ID NO: 10) | [(GAGAGS)₉RYVVLPRPVCFEKAAGY]ₙ |
| ELP I | (SEQ ID NO: 11) | [(VPGVG)₄]ₙ |
| SELP 0 | (SEQ ID NO: 12) | [(GVGVP)₈(GAGAGS)₂]ₙ |
| SELP 1 | (SEQ ID NO: 13) | [GAA(VPGVG)₄VAAGY(GAGAGS)₉]ₙ |
| SELP 2 | (SEQ ID NO: 14) | [(GAGAGS)₆GAAGY(GAGAGS)₈(GVGVP)₈]ₙ |
| SELP 3 | (SEQ ID NO: 15) | [(GVGVP)₈(GAGAGS₈]ₙ |
| SELP 4 | (SEQ ID NO: 16) | [(GVGVP)₁₂(GAGAGS)₈]ₙ |
| SELP 5 | (SEQ ID NO: 17) | [(GVGVP)₁₆(GAGAGS)₈]ₙ |
| SELP 6 | (SEQ ID NO: 18) | [(GVGVP)₃₂(GAGAGS)₈]ₙ |
| SELP 7 | (SEQ ID NO: 19) | [(GVGVP)₈(GAGAGS)₆]ₙ |
| SELP 8 | (SEQ ID NO: 20) | [(GVGVP)₈(GAGAGS)₄]ₙ |
| KLP 1.2 | (SEQ ID NO: 21) | [(AKLKLAEAKLELAE)₄]ₙ |
| CLP 1 | (SEQ ID NO: 22) | [GAP(GPP)₄]ₙ |
| CLP 2 | (SEQ ID NO: 23) | {[GAP(GPP)₄]₂GPAGPVGSP}ₙ |
| CLP-CB | (SEQ ID NO: 24) | {[GAP(GPP)₄]₂(GLPGPKGDRGDAGPKGADGSPGPA)GPAGPVGS-P}ₙ |
| CLP 3 | (SEQ ID NO: 25) | (GAPGAPGSQGAPGLQ)ₙ |

Repetitive amino acid sequences of selected protein polymers. SLP=silk like protein; SLPF=SLP containing the RGD sequence from fibronectin; SLPL 3/0 and SLPL 3/1=SLP containing two difference sequences from laminin protein; ELP=elastin like protein; SELP=silk elastin like protein; CLP=collagen like protein; CLP−CB=CLP containing a cell binding domain from human collagen; KLP=keratin like protein.

Engineered proteins may also include therapeutic peptide fragments to provide therapeutic benefit to a patient when the proteins are used, for example, as lenses. In addition to aECM-RGD, embodiments of engineered proteins that may be used include, but are not limited to, other suitable engineered proteins with a cell-binding or elastic domain.

In exemplary embodiments of the disclosure, aECM is mixed with a polymer crosslinker in situ to form a protein-polymer hydrogel as the adhesive bond. The term “polymer crosslinker” refers to molecules that are polymeric (comprising 10 or more repeat units) and have two or more reactive sites that are capable of forming a covalent or physical bond with at least one amino acid residue present in aECM and at least one amino acid residue of structural proteins (such as collagens). The number of reactive sites on each polymer crosslinker is defined as its “valency.” The polymer crosslinker may be linear or branched or dendrimeric. Branched architectures include those known in the polymer literature as star, H, comb, and hyperbranched architectures. The repeat units can be chosen from those known to be well tolerated in the body, such as ethylene glycol.

In one of the preferred embodiments of the disclosure, the protein aECM-RGD is mixed in situ with the polymer crosslinker PEG-S. The PEG-S polymer crosslinker is a four-arm telechelic polyethylene glycol with succinimidyl glutarate end groups. FIG. 2 is an illustration of a chemical structure 12 of PEG-S polymer crosslinker. The succinimidyl glutarate end groups form covalent linkages with primary amines on both aECM and structural tissues proteins to create the adhesive bond. The polyethylene glycol backbone is preferable due to its low toxicity. The N-hydroxysuccinimide (NHS) is a good leaving group, and the NHS-ester moieties found on each arm of PEG-S can readily react with nucleophilic groups to form covalent linkages. This reaction has been demonstrated in known tissue adhesives (Wallace et al., “A Tissue Sealant Based on Reactive Multifunctional Polyethylene Glycol”, Journal of Biomedical Materials Research Part B: Applied Biomaterials, 58:545-555 (2001)), and in the crosslinking reaction to form corneal onlays with aECM-RGD (Nowatzki P. J., “Characterization of Crosslinked Artificial Protein Films”, Doctoral Thesis, California Institute of Technology, Pasadena, Calif. 2006). The use of PEG-S in other tissue adhesives has also been shown and that the compound is biocompatible in vascular repair and subcutaneous implants in rabbits (Wallace et al., “A Tissue Sealant Based on Reactive Multifunctional Polyethylene Glycol”, Journal of Biomedical Materials Research Part B: Applied Biomaterials, 58:545-555 (2001)). In disclosed embodiments of the disclosure, PEG-S is mixed with aECM-GD, where the NHS-esters react with primary amines found on lysine and arginine residues of the artificial protein and native proteins in the tissues. The resulting amide bonds form a covalent network that provides the mechanical strength necessary for the adhesive bond. In addition, it has been shown that cells preferentially bind to aECM-covered areas over adjacent PEG-covered areas (Carrico et al., “Lithographic Patterning of Photoreactive Cell-Adhesive Proteins”, JACS Communications, J. Am. Chem. Soc., 129:4874-4875 (2007)).

Standard protein expression and purification procedures can be used to synthesize aECM-RGD (Nowatzki P. J., “Characterization of Crosslinked Artificial Protein Films”, Doctoral Thesis, California Institute of Technology, Pasadena, Calif., 2006). PEG-S can be obtained at low polydispersity from commercial vendors, or synthesized by anionic living polymerization and subsequent end group modification. Certain embodiments of the disclosure involve dissolving aECM-RGD and PEG-S into separate aqueous solutions. These two solutions can be premixed and then applied to wound sites where polymerization occurs. For slowly gelling formulations, the two solutions can be premixed, while rapidly gelling formulations may require a two-component applicator or spray device.

One with skill in the art realizes that the two solid compounds may be dissolved in buffers of varying pH in order to modulate gelling time or mechanical strength. In addition, the compounds can be dissolved in the same solution but maintained at a pH unfavorable for reaction. Polymerization can then be activated by changing the pH environment of the compounds.

One with skill in the art realizes that crosslinkers of different size, valency, or even fundamental chemistry are also possible in place of PEG-S. One with skill in the art realizes that adjusting the size and valency of the polymer crosslinker can give a variety of mechanical strengths, and that different crosslinking chemistry can be used to allow adhesion at different reaction conditions. For example, tissue adhesives in the literature use peptide ligation chemistry (Grinstaff M. W., “Designing Hydrogel Adhesives for Corneal Wound Repair”, Biomaterials, 28:5205-5214 (2007)), or Maillard reactions (U.S. Pat. No. 7,129,210 to Lowinger et al., entitled “Tissue Adhesive Sealant”). Additional crosslinking chemistries that are known in the art include Michael addition (Lutolf et al., “Synthesis and Physicochemical Characterization of End-linked Poly(ethylene glycol)-co-peptide Hydrogels Formed by Michael-type Addition”, Biomacromolecules, 4:713-722 (2003)), and thiazolidine formation (Grinstaff M. W., “Designing Hydrogel Adhesives for Corneal Wound Repair”, Biomaterials, 28:5205-5214 (2007)).

The crosslinkers preferably contain multiple (two or more) functional groups per molecule that are compatible with multiple (two or more) crosslinking sites in the protein sequence. The crosslinking chemistry can be changed by choice of the reactive groups of the polymer crosslinker or by engineering the aECM artificial protein. For example, photopolymerizable moieties may be tethered to aECM to give a photoactivated adhesive.

In addition to PEG-S, other suitable polymer crosslinkers may be used. The engineered proteins may be crosslinked by reacting the proteins with a suitable and biocompatible crosslinking agent. The engineered protein may be crosslinked by utilizing methods generally known in the art. For example, the engineered protein may be partially or entirely crosslinked by exposing, contacting and/or incubating the engineered protein device with a liquid crosslinking reagent, light, or combination thereof.

An adjustable biomedical implant, for example, a corneal implant, may be prepared from the engineered protein by including reactive side chains that are susceptible to photochemical crosslinking. Attachment of acryloyl or methacryloyl groups to the lysine side chains of the protein yields photocurable variants that can be crosslinked by laser irradiation. Inclusion of low molecular weight proteins, similarly functionalized, provides a basis for changing the local curvature of the implant through patterned irradiation and diffusion of low molecular weight species in response to an osmotic gradient. After the intended shape change is accomplished, the structure is “locked” by further irradiation of the entire implant.

The use of PEG-S in other FDA (U.S. Food and Drug Administration)-approved tissue adhesives has shown that the compound is biocompatible in subcutaneous rabbit implants and in human vascular repair (Wallace et al., “A Tissue Sealant Based on Reactive Multifunctional Polyethylene Glycol”, Journal of Biomedical Materials Research Part B: Applied Biomaterials, 58:545-555 (2001); CoSeal Surgical Sealant, U.S. FDA approval P030039, Dec. 12, 2003). Introduction of both PEG-S and aECM into tissues can initiate an in situ crosslinking reaction where the NHS moieties form covalent linkages with primary amines found on aECM lysine residues and on structural tissue proteins. The resulting covalent network gel is mechanically strong while still incorporating the favorable cell-adhesive properties of aECM.

The PEG-S/aECM formulation may also be applied as a film on the anterior surface of the cornea, providing artificial protection for those with diseases of the epithelium. Additionally, this adhesive can also be used in other tissue applications, especially ones where a thin cell layer provides function to tissues. One such application is vascular grafts, where the endothelium prevents thrombosis and secretes molecules to regulate the vascular environment (Wallace et al., “A Tissue Sealant Based on Reactive Multifunctional Polyethylene Glycol”, Journal of Biomedical Materials Research Part B: Applied Biomaterials, 58:545-555 (2001)).

Additional applications of one or more embodiments of the tissue adhesive of the disclosure may include, but are not limited to, sealing corneal ulcers and perforations, reducing or eliminating the need for sutures in keratoplasties, adhering corneal onlays onto the stroma for vision correction, correcting refractive errors, and reattaching LASIK (laser-assisted in situ keratomileusis) flaps. Additionally, one with skill in the art realizes that one or more embodiments of the tissue adhesive of the disclosure can be used in other areas besides the cornea. For example, one or more embodiments of the tissue adhesive of the disclosure has advantages over other adhesives wherever a thin cell layer provides function to tissues. One such application is vascular grafts, where the endothelium prevents thrombosis and secretes molecules to regulate the vascular environment (Heilshorn et al., “Endothelial Cell Adhesion to the Fibronectin CS5 Domain in Artificial Cellular Matrix Proteins”, Biomaterials, 24:4245-4252 (2003)).

In one embodiment of the disclosure, there is provided a tissue adhesive composition comprising an engineered protein having repeated blocks of an elastin domain and at least one cell-binding domain, and further comprising a polymer crosslinker. The engineered protein and the polymer crosslinker are kept separate prior to application. When the engineered protein and the polymer crosslinker are introduced onto a tissue, the engineered protein and the polymer crosslinker initiate an in situ crosslinking reaction to form an adhesive bond that is mechanically strong, transparent, biocompatible, and stimulates regrowth of one or more tissue layers over the adhesive bond. Preferably, the engineered protein is aECM-RGD comprising SEQ ID NO: 1, or another suitable engineered protein. Preferably, the elastin domain comprises one of SEQ ID NO: 2-4, 11, 12-19, or 20. Preferably, the cell-binding domain comprises a fibronectin domain comprising one of SEQ ID NO: 26 or 27. Preferably, the polymer crosslinker may comprise a linear telechelic PEG (polyethylene glycol), a star PEG (polyethylene glycol) with two or more arms, or other suitable polymer crosslinkers. More preferably, the polymer crosslinker may comprise a four-arm polyethylene glycol with succinimidyl glutarate end groups (PEG-S). Preferably, the engineered protein and the polymer crosslinker are present in a ratio of about 1:1, such as a 1:1 ratio of lysine residues to succinimidyl glutarate end groups. However, other suitable ratios may also be used, depending on, for example, the number of lysines in a sequence or the number of arms in a PEG molecule. Preferably, the engineered protein is present in an amount of from about 10% weight per volume (w/v) to about 40% weight per volume (w/v) based on the total weight per volume of the tissue adhesive composition. Preferably, the polymer crosslinker is present in an amount of from about 10% weight per volume (w/v) to about 40% weight per volume (w/v) based on the total weight per volume of the tissue adhesive composition. Preferably, the tissue adhesive composition comprises a corneal adhesive for use in a mammalian eye.

The tissue adhesive composition may also be in the form of a molded corneal onlay in a mammalian eye. Preferably, the tissue adhesive composition has applications such as sealing corneal ulcers and perforations, reducing or eliminating the need for sutures in keratoplasties, adhering corneal onlays onto a stroma for vision correction, reattaching LASIK (laser-assisted in situ keratomileusis) flaps, correcting refractive errors, and/or providing vascular tissue grafts.

In one embodiment, there is provided a method to provide a tissue adhesive composition. The method comprises combining an engineered protein and a polymer crosslinker, the engineered protein comprising repeated blocks of an elastin domain and at least one cell-binding domain. Preferably, the combining is performed by providing the engineered protein in about 10% weight per volume (w/v) to about 40% weight per volume (w/v) based on the total weight per volume of the resulting tissue adhesive composition and combining the engineered protein with the polymer crosslinker. Preferably, the engineered protein comprises aECM-RGD comprising SEQ ID NO: 1. Preferably, the combining is performed by providing about 10% weight per volume (w/v) to about 40% weight per volume (w/v) of a polymer crosslinker based on the total weight per volume of the resulting tissue adhesive composition and combining the polymer crosslinker with the engineered protein, possibly also provided in the above mentioned range. Preferably, the polymer crosslinker may comprise a linear telechelic PEG (polyethylene glycol), a star PEG with two or more arms, or another suitable polymer crosslinker. More preferably, the polymer crosslinker may comprise a four-arm polyethylene glycol with succinimidyl glutarate end groups (PEG-S).

In one embodiment, there is provided a system to provide a tissue adhesive composition. The system comprises an engineered protein and a polymer crosslinker, the engineered protein comprises repeated blocks of an elastin domain and at least one cell-binding domain. Preferably, the polymer crosslinker may comprise a linear telechelic PEG (polyethylene glycol), a star PEG with two or more arms, or another suitable polymer crosslinker. More preferably, the polymer crosslinker may comprise a four-arm polyethylene glycol with succinimidyl glutarate end groups (PEG-S). Preferably, the tissue adhesive composition comprises a corneal adhesive for use in a mammalian eye. When the engineered protein and the polymer crosslinker are introduced onto a tissue, the engineered protein and the polymer crosslinker initiate an in situ crosslinking reaction to form an adhesive bond that is mechanically strong, optically transparent, biocompatible, and stimulates regrowth of one or more tissue layers over the adhesive bond.

In another embodiment of the disclosure, there is provided a molded corneal onlay for use in a mammalian eye. The molded corneal onlay may comprise a bulk hydrogel comprising an engineered protein having repeated blocks of an elastin domain and at least one cell-binding domain, and further comprising a polymer crosslinker. The bulk hydrogel may be molded on a corneal surface to form a molded corneal onlay, and the engineered protein and the polymer crosslinker initiate an in situ crosslinking reaction to attach the molded corneal onlay to the corneal surface. In one embodiment, the molded corneal onlay can be molded in vitro, e.g., on a corneal tissue or on an artificial surface mimicking a corneal surface, before application on the corneal surface in a human or animal body. In one embodiment, the molded corneal onlay can be molded in vivo, e.g., directly on the corneal surface of the individual where the molded corneal onlay is finally applied. The molded corneal onlay is preferably optically transparent, biocompatible, protects the corneal surface, and stimulates cellular regrowth of corneal cells. In addition, the corneal onlay may be used for correcting refractive errors. Preferably, the engineered protein is aECM-RGD comprising SEQ ID NO: 1, or another suitable engineered protein. Preferably, the polymer crosslinker may comprise a linear telechelic PEG (polyethylene glycol), a star PEG (polyethylene glycol) with two or more arms, or other suitable polymer crosslinkers. More preferably, the polymer crosslinker may comprise a four-arm polyethylene glycol with succinimidyl glutarate end groups (PEG-S).

In another embodiment of the disclosure, there is provided a method of adhering tissue. The method comprises applying a tissue adhesive composition to one or more tissue surfaces in vitro and/or in vivo. The tissue adhesive composition comprises an engineered protein having repeated blocks of an elastin domain and at least one cell-binding domain, and further comprises a polymer crosslinker. When the engineered protein and the polymer crosslinker are applied to the one or more tissue surfaces, the engineered protein and the polymer crosslinker initiate an in situ crosslinking reaction to form an adhesive bond. In one embodiment, the engineered protein and polymer crosslinker are applied to the one or more tissue surfaces in vitro, possibly before applying the tissue surfaces comprising the engineered protein and polymer crosslinker on a human or animal body. In one embodiment, the engineered protein and polymer cross-linker are applied to the one or more tissue surfaces in vivo, e.g., directly on a human or animal tissue such as a cornea. The method further comprises curing the tissue adhesive composition to bond the composition to the one or more tissue surfaces and to provide a cured adhesive bond that is mechanically strong, transparent, biocompatible, and stimulates regrowth of one or more tissue layers over the cured adhesive bond. In one embodiment the curing is performed in vitro possibly before applying the cured tissue surface on the human or animal body. In one embodiment, the curing is performed on a tissue in vivo. Preferably, the tissue adhesive composition comprises about 10% weight per volume (w/v) to about 40% weight per volume (w/v) of an engineered protein based on the total weight per volume of the tissue adhesive composition. Preferably, the engineered protein comprises aECM-RGD comprising SEQ ID NO: 1, or another suitable engineered protein. Preferably, the tissue adhesive composition further comprises about 10% weight per volume (w/v) to about 40% weight per volume (w/v) of the polymer crosslinker based on the total weight per volume of the tissue adhesive composition. Preferably, the polymer crosslinker may comprise a linear telechelic PEG (polyethylene glycol), a star PEG (polyethylene glycol) with two or more arms, or other suitable polymer crosslinkers. More preferably, the polymer crosslinker may comprise a four-arm polyethylene glycol with succinimidyl glutarate end groups (PEG-S).

In one embodiment of the disclosure, there is provided a system for adhering a tissue. The system comprises a tissue adhesive composition and at least one tissue substrate for simultaneous sequential or separate use in a method of adhering tissue herein described. In one embodiment, the system comprises two or more tissue substrates and at least one of the applying and the curing is performed in vitro.

In another embodiment of the disclosure, there is provided a method of making a molded corneal onlay for use in a mammalian eye. The method comprises providing a bulk hydrogel comprising an engineered protein having repeated blocks of an elastin domain and at least one cell-binding domain, and further comprising a polymer crosslinker. The method further comprises molding the bulk hydrogel on a corneal surface to form a molded corneal onlay. In one embodiment, the molded corneal onlay can be molded in vitro, e.g., on a corneal tissue substrate or on an artificial surface mimicking a corneal surface, before application on the corneal surface in a human or animal body. In one embodiment, the molded corneal onlay can be molded in vivo, e.g., directly on the corneal surface of the individual where the molded corneal onlay is finally applied. The method further comprises attaching the molded corneal onlay to the corneal surface via the engineered protein and the polymer crosslinker initiating an in situ crosslinking reaction. In particular, in one embodiment, attaching can be performed in vitro, e.g., on a corneal surface outside a human or animal body. In one embodiment, the attaching can be performed in vivo and in particular, on a corneal surface of an individual to which the molded corneal onlay is attached. In one embodiment, the molded corneal onlay can be used in a treatment of a cornea. In one embodiment, the molded corneal onlay can, in particular, be used in a treatment of a corneal implant. The molded corneal onlay formed is optically transparent, biocompatible, protects the corneal surface, is used to correct refractive errors, and stimulates cellular regrowth of corneal cells. Preferably, the engineered protein is aECM-RGD comprising SEQ ID NO: 1. Preferably, the polymer crosslinker may comprise a linear telechelic PEG (polyethylene glycol), a star PEG (polyethylene glycol) with two or more arms, or other suitable polymer crosslinkers. More preferably, the polymer crosslinker may comprise a four-arm polyethylene glycol with succinimidyl glutarate end groups (PEG-S).

In one embodiment of the disclosure, there is provided a system for providing a molded corneal onlay for use in a mammalian eye. The system comprises a bulk hydrogel and a corneal tissue substrate for simultaneous sequential or separate use in a method of making a molded corneal onlay herein described wherein at least one of the molding and the attaching is performed in vitro.

In one embodiment, the systems herein disclosed can be provided in the form of a kits of parts. In a kit of parts, the engineered protein, polymer crosslinker, bulk hydrogel and/or tissue substrate are comprised in the kit independently. The engineered protein and polymer crosslinker can be included in one or more compositions, and each engineered protein and/or polymer crosslinker may be in a composition together with a suitable vehicle carrier or auxiliary agent.

In some embodiments, a crosslinking agent can be further provided as an additional component of the kit. Additional components can include reference standards, additional reagents and additional components identifiable by a skilled person upon reading of the present disclosure. In particular, the components of the kit can be provided, with suitable instructions and other necessary reagents, in order to perform the methods herein disclosed. The kit will normally contain the compositions in separate containers. Instructions, for example, written or audio instructions, on paper or electronic support, such as tapes or CD-ROMs, for carrying out the assay, will usually be included in the kit. The kit can also contain, depending on the particular method used, other packaged reagents and materials (i.e. wash buffers and the like).

Further details concerning the identification of the suitable carrier agent or auxiliary agent of the compositions, and generally manufacturing and packaging of the kit, can be identified by the person skilled in the art upon reading of the present disclosure.

### EXAMPLES

The following examples are intended to describe and illustrate the practice of the disclosed embodiments. The examples, however, should not be construed to limit the scope of the disclosure which is defined by the appended claims.

PEG-S. For the examples, PEG-S was obtained from Polymer Source, Inc. of Montreal, Canada, and stored at −20° C. (degrees Celsius) with a desiccant until use. It should be noted that the NHS-ester (N-Hydroxysuccinimide-ester) moieties slowly degrade upon contact with moisture. Thus, solutions were prepared in deionized water at most three (3) hours before use. The received lot of PEG-S formed a very cloudy solution, suggesting the presence of impurities. For experiments that required optical clarity, the solutions were centrifuged at 16,000 g (gravity) for ten (10) minutes, and then the supernatant was collected and centrifuged again at 16,000 g for ten (10) minutes. Known assays (Miron et al., “A Spectrophotometric Assay for Soluble and Immobilized N-hydroxysuccinimide Esters”, Analytic Biochemistry, 126:433-435 (1982)) showed the activity of the PEG-S was unchanged by this purification step.

aECM expression and purification. The aECM material was synthesized by known methods (Nowatzki P. J., “Characterization of Crosslinked Artificial Protein Films”, Doctoral Thesis, California Institute of Technology, Pasadena, Calif., 2006). The gene of interest was inserted into the pET-28a vector, confirmed by sequencing, and then transformed into the expression host BL21 (DE3) pLysS (obtained from EMD Chemicals, Inc. of Gibbstown, N.J.). For all expressions, Terrific Broth medium made in the lab was used with 25 mg/L (milligrams per liter) of kanamycin and 35 mg/L of chloramphenicol at 37° C. Two different protocols were used for protein expression, depending on desired yield. For small expressions, cells were grown overnight in a 5 mL (milliliter) culture, which was used to inoculate 1 L (liter) of media. The 1 L cultures were shaken in a 2.8 L Fernbach-style culture flask. At an optical density (absorbance at 600 nm (nanometers)) of approximately one (1), expression was induced with 1 mM (millimolar) isopropyl-1-β-D-thiogalactosidase (IPTG) reagent. Cells were harvested five (5) hours after induction by centrifugation at 11,000 g for ten (10) minutes at 4° C. Approximately 7 g (grams) of wet cell mass was obtained for each 1 L culture. For large expressions, cells were grown overnight in a 5 mL culture, which inoculated 1 L of media, which inoculated a total 10 L of media in a BIOFLO Pro 3000 liter fermentor, obtained from New Brunswick Scientific Co., Inc., of Edison, N.J. (BIOFLO is a registered trademark of New Brunswick Scientific Co., Inc., of Edison, N.J.). Air flow was controlled through a sparger and pH was maintained at 7.4. At an optical density of approximately six (6), expression was induced with 1 mM IPTG. Cells were harvested 2 to 3.5 hours after induction by centrifugation. Approximately 240 g (grams) of wet cell mass was obtained for each 10 L fermentation.

Harvested cells were resuspended in TEN buffer (10 mM Tris, 1 mM EDTA (ethylenediaminetetraacetic acid), 100 mM (millimolar) NaCl (sodium chloride), pH 7.5) at a concentration of 0.3 g/mL (grams per milliliter) or less. The suspension was sonicated and frozen at −80° C. overnight. Ten (10) μg/mL (micrograms per milliliter) of DNAase I, 10 μg/mL of RNAase A, 5 mM of MgCl (magnesium chloride), and 1 mM (millimolar) of phenylmethylsulfonyl fluoride were added while the suspension was defrosting. The partially defrosted solution was then shaken at 37° C. for three (3) hours. Since the aECM protein exhibited a lower critical solution temperature (LCST) of 35° C., temperature cycling was used for purification. The pH of the cell lysate was adjusted to 9.0, and the solution was stirred at 4° C. for at least six (6) hours to insure protein solubility. The solution was then centrifuged at greater than or equal to (≧)30,000 g for two (2) hours at 4° C. To the supernatant (containing the protein), 1M NaCl was added, and the solution was shaken at 37° C. for at least six (6) hours to maximize protein precipitation. Centrifugation was repeated at greater than or equal to (≧)30,000 g for two (2) hours at 40° C. The pellet was resuspended in water at less than (<)50 g/L (grams per liter), and the centrifuge cycles were repeated at least twice more. The final solution was loaded into dialysis tubing obtained from Spectrum Laboratories, Inc. of Rancho Dominguez, Calif., (6-8 kDa (kilo Dalton) molecular weight cutoff) at 4° C. for three (3) days and lyophilized. The buffer exchange was done manually. The resulting pure protein was confirmed by SDS-PAGE (sodium dodecyl sulfate polyacrylamide gel electrophoresis) and MALDI-TOF (Matrix Assisted Laser Desorption/Ionization-Time Of Flight) mass spectrometry. One (1) L cultures yielded approximately 8 mg-40 mg (milligrams) and 10 L cultures yielded approximately 2 g (grams) of pure protein.

Tissue substrates. Common tissue substrates were used in a variety of examples. Gelatin was often used as a model proteinaceous tissue. 300 bloom type A gelatin powder from porcine skin, obtained from Sigma-Aldrich of St. Louis, Mo., was used as received. This was added to Dulbecco's phosphate buffered saline (PBS) in a 15% (w/v (weight per volume)) proportion. The mixture was heated to 65° C. while stirring in order to melt the powder, and then molded into an approximately 1 mm (millimeter) thick film. The molds were cooled at 4° C. for one to five (1-5) days before use.

For in vitro experiments with intact eyes or corneal tissue, porcine eyes were obtained from Siena for Medical Science (Santa Fe Springs, Calif., USA). The animals were five to six (5-6) months old and weighed approximately 100 kg (kilograms). Specimens were refrigerated during shipping and storage, and used within two (2) days post-mortem. For all experiments, the corneal epithelium was mechanically debrided from the intact eyes using a scalpel blade. In studies requiring the corneal tissue only, the cornea and 1 mm-2 mm of the adjacent corneo-scleral limbus were removed from the globe. These intact corneo-scleral buttons were stored in a humidified environment and tested within four (4) hours of dissection.

### Example 1

In vivo study of Epithelium Regrowth over an aECM-RGD Film. Previous studies have demonstrated that a crosslinked film of aECM-RGD promotes epithelium growth over the protein surface (Nowatzki P. J., “Characterization of Crosslinked Artificial Protein Films”, Doctoral Thesis, California Institute of Technology, Pasadena, Calif., 2006). This film is a corneal onlay, formed using the following procedure. Twenty (20) mg (milligrams) of aECM-RGD were mixed with 2.3 mg of BS3 (Bis(Sulfosuccinimidyl) suberate), a bifunctional sulfo-NHS-ester that crosslinks amines similar to PEG-S, in 75 μL (microliters) of water. Twelve (12) μL of this mixture was pipetted into a disk-shaped mold of 6 mm (millimeters) diameter and 135 μm (micrometers) thickness, and allowed to crosslink overnight at 4° C. The corneal onlay was then implanted in vivo into a stromal pocket of a rabbit cornea, as discussed in the literature. FIG. 10A shows a clinical exam photograph depicting results from the in vivo study of epithelium regrowth over an aECM-RGD film on a rabbit cornea immediately after implantation (t (time)=0 (zero)). FIG. 10B shows a clinical exam photograph depicting results from the in vivo study of epithelium regrowth over an aECM-RGD film on a rabbit cornea two (2) days after implantation (t (time)=2 (two) days)). FIG. 10C shows a clinical exam photograph depicting results from the in vivo study of epithelium regrowth over an aECM-RGD film on a rabbit cornea seven (7) days after implantation (t (time)=7 (seven)). In comparison, placebo tests where no onlays were implanted showed complete re-epitheliazation within four (4) days.

Since crosslinked films of aECM-RGD were found to be effective in promoting epithelium regrowth, disclosed embodiments applied over a wound site were expected to provide similarly favorable results. Furthermore, since these films can be made sufficiently transparent to form corneal onlays, disclosed embodiments were expected to retain similar optical clarity properties.

### Example 2

Mechanical Strength of Adhesives. A custom method was developed to evaluate and compare the adhesive strength of the PEG-S/aECM mixture. Many ophthalmic applications require adhesives with sufficient shear strength. For example, in applying corneal onlays and reattaching LASIK (laser-assisted in situ keratomileusis) flaps, shear forces will be the primary load on the adhesive bond. Accordingly, a shear rheometer was developed to quantitatively demonstrate the mechanical strength of adhesive bonds formed by embodiments of the disclosure. With the custom method, gelatin disk or tissue specimens were mounted on the head plate and base plate of a shear rheometer device, an adhesive was applied between the gelatin or tissue specimens, and the specimens were brought together. A linearly increasing torque was applied to the head plate until the adhesive bond failed. Strength was assigned as the torque at failure marked by a sharp rise in velocity.

For this example, gelatin (preparation described above) was used as a model for human tissue or crosslinked onlays. This substitution was believed to be valid since gelatin, tissue, and onlays are all highly hydrated protein films. Further, such substitution was advantageous since gelatin is inexpensive and easy to prepare. For comparison with real applications, corneal tissue (preparation described above) was also used in this study. In summary, adhesives were tested between two pieces of gelatin, or between a piece of gelatin and a piece of corneal stroma.

FIG. 3 is an illustration of a front view in partial cross-section of a shear rheometer 20 that was used for testing shear adhesive strength of various tissue samples. A TA Instruments stress-controlled Advanced Rheometer (AR1000) was used for this study. As shown in FIG. 3, a first disk of gelatin or corneal tissue 22 was attached to a base plate 24 of the rheometer 20 with a cyanoacrylate glue. However, another suitable attachment or adhesive means may be used. A second disk of gelatin 28 was mounted to a head plate 30 of the rheometer 20 with a cyanoacrylate glue. However, another suitable attachment or adhesive means may be used. The head plate 30 was aluminum. However, the head plate may be made of another suitable metal or material. The head plate 30 had a diameter of 8 mm (millimeters). However, the head plate may have another suitable size diameter. The head plate 30 was attached to a central spindle 32 of the rheometer 20 where the central spindle 32 was capable of rotating. The second disk of gelatin 28 had a diameter of 8 mm and a thickness of 1 mm. However, the second disk of gelatin may have another suitable size diameter or another suitable size thickness. Since the first disk of gelatin 22 was larger than the second disk of gelatin 28, the contact area of the adhesive was dictated by the size of the second disk only.

Many adhesive formulations were tested for comparison in this example. In all cases, the adhesive was applied to one or both of the two mounted tissues (22 or 28), the head plate 30 was lowered to a normal force of about 0.1N (newtons), and the entire apparatus was undisturbed to allow the adhesive to cure. The torque (t) of the rheometer 20 was then increased from 0 to 10⁴ μN-m (micronewton-meters) in a time span of ten (10) minutes. Strength was assigned as the torque at the failure (marked by a sharp rise in velocity). The experiments were all performed in open air at 25° C.

“PEG-S/aECM” was one test condition which was an embodiment of an adhesive of the disclosure. This was prepared by dissolving 20 mg (milligrams) of aECM-RGD in 55 μL (microliters) of water, and dissolving 10 mg of PEG-S in 400 μL of water. Equal volumes of the two solutions were premixed in a microcentrifuge tube and spun down to eliminate bubbles. Approximately 5 μL of the adhesive was applied immediately following premixing between the two model tissues. As shown in FIG. 3, PEG-S/aECM adhesive 26 is sandwiched between the first disk of gelatin 22 and the second disk of gelatin 28. The adhesive was cured for twenty (20) minutes.

An additional test condition was DERMABOND, an adhesive obtained from Ethicon, Inc. of Somerville, N.J. (DERMABOND is a registered trademark of Johnson & Johnson Corporation of Brunswick, N.J.). DERMABOND is a cyanoacrylate adhesive for topical application of skin wounds, representative of a large family of adhesives based on the same chemistry (Vote et al., “Cyanoacrylate Glue for Corneal Perforations: A Description of a Surgical Technique and a Review of the Literature”, Clinical and Experimental Opthalmology, 28:437-442 (2000); Bernard et al., “A Prospective Comparison of Octyl Cyanoacrylate Tissue Adhesive (DERMABOND) and suture for the closure of excisional wounds in children and adolescents”, Archives of Dermatology, 137:1177-1180 (2001)). Ten (10) μL of DERMABOND was applied between the model tissues and allowed to cure for five (5) minutes. Testing began after fifteen (15) minutes of curing.

An additional test condition was PBS (phosphate buffered saline) which was also tested as an adhesive to provide a control. Ten (10) μL of PBS was applied between the model tissues as a control.

An additional test condition was “Dry”. “Dry” denoted no liquid between the model tissues, and was also used as a control.

An additional test condition was “PEG sealant”. This adhesive was derived from COSEAL obtained from Baxter International, Inc. of Deerfield, Ill. (COSEAL is a registered trademark of Baxter International, Inc. of Deerfield, Ill.). COSEAL is a polymer adhesive that utilizes PEG-S along with PEG-T, which is structurally similar to PEG-S except thiols replace the NHS moieties (Wallace et al., “A Tissue Sealant Based on Reactive Multifunctional Polyethylene Glycol”, Journal of Biomedical Materials Research Part B: Applied Biomaterials, 58:545-555 (2001)). This formulation has been used as a sealant in tandem with sutures in vascular repair. The two-component crosslinking of PEG sealant is controlled by pH, so it was necessary to modify the usual application of COSEAL to ensure proper curing as the specimens are brought into contact. In COSEAL, PEG-S and PEG-T are premixed in a low pH buffer; polymer crosslinking is initiated in a high pH environment. To initiate curing when the specimens are brought into contact, the pH of the gelatin and cornea surfaces was raised. Gelatin was modified by preparation with PC9 buffer (total of 300 mM NaH₂PO₄ and Na₂CO₃ in water, pH=9). The stroma was modified by applying 10 μL of PC9 buffer to the anterior surface and allowing ten (10) minutes for diffusion to occur before removing excess fluid using KIMWIPES. (KIMWIPES is a registered trademark of Kimberly-Clark Corporation of Neenah, Wis.). For testing, 10 μL of the low pH COSEAL component (containing the polymers) was applied to the gelatin on the top fixture 28 and then the surfaces were brought together. Curing time was fifteen (15) minutes for all PEG sealant tests.

Table 2 shows results from the shear rheometry procedure applied to PBS, DERMABOND, and PEG-S/aECM adhesive. The numbers listed were the torques observed at 95% confidence intervals. Failures for PBS and PEG-S/aECM adhesive occurred at the adhesive bond, while failures for DERMABOND occurred in the bulk gelatin material.

TABLE 2

| | Adhesive | | |
|---|---|---|---|
| | PBS (Control) | DERMABOND | PEG-/aECM |
| Strength | 410 ± 40 | 2500 ± 360 | 2200 ± 460 |
| (μN-m) | | | |

The results clearly showed that PEG-S/aECM adhesive exhibited an impressive adhesive strength. This was evident from the large difference between the strength of PBS versus the strength of PEG-S/aECM adhesive. Further, the strength of 2500 μN-m as listed for DERMABOND defined the maximum strength measurable in the gelatin model because this corresponded to the torque at bulk material failure. The PEG-S/aECM adhesive gave strengths similar to this threshold, and thus provided evidence that indicated an adhesive of considerable shear strength.

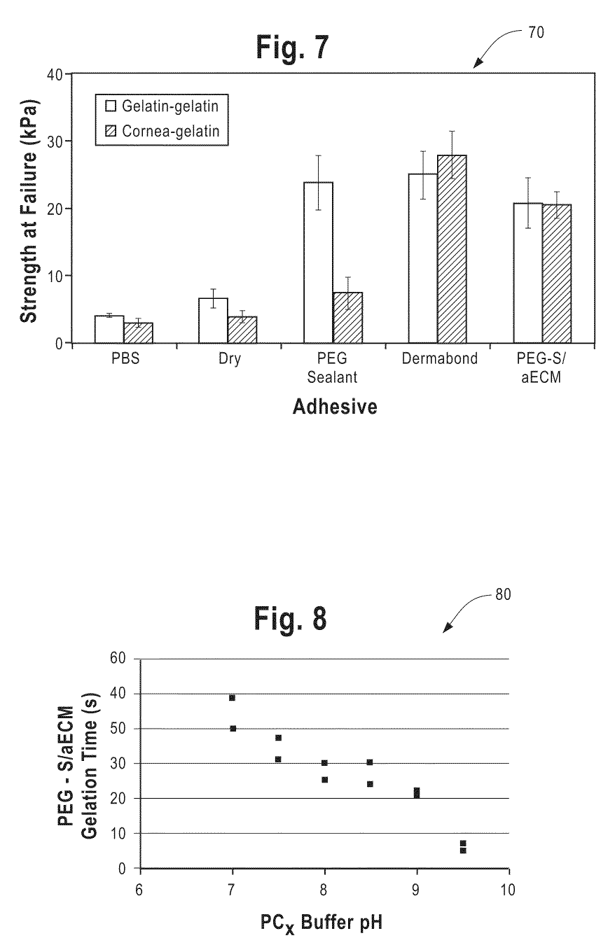

FIG. 7 is a bar graph 70 showing shear stress strength at failure (kPa (kilopascal)) compared between adhesives PBS, Dry, COSEAL, DERMABOND, and PEG-S/aECM adhesive. Eight (8) runs were performed for each test condition, and error bars depicted 95% confidence intervals according to a Student's t-test statistic. Nine (9) runs were performed for PEG-S/aECM on gelatin-gelatin, and six (6) runs were performed on PEG-S/aECM on stroma-gelatin.

It is important to note that failure can occur by cohesive failure (fracture in the bulk hydrogel) or adhesive failure (fracture at the adhesive bond). The observed failure stress was the smaller of these two (i.e., the one that occurs first as torque increases). For PEG sealant in the gelatin-gelatin configuration, adhesive failure occurred in five (5) of the eight (8) tests, suggesting that the measured value was close to both the bulk strength of gelatin and the actual adhesive strength. The adhesive strength of PEG sealant in the cornea-gelatin bond was less than the gelatin-gelatin case, presumably due to the different application and curing procedure. In the DERMABOND tests for both gelatin-gelatin and cornea-gelatin, cohesive failure was observed. Since the fracture of gelatin always occurred at 25 kPa-30 kPa (kilopascal), these measurements were limited by the bulk strength of gelatin.

As expected, the failure strengths of PBS and Dry were very low compared to that of the other adhesives. PEG sealant, DERMABOND, and PEG-S/aECM all indicated similar failure strengths in the 20 kPa-25 kPa (kilopascal) range. Since more than half of the failure events in PEG sealant were at the adhesive bond, it was concluded that the embodiments of the adhesive of the disclosure was comparable to COSEAL in strength. Indeed, adhesive formulations of PEG-S/aECM can readily be compared to other adhesives in the literature, such as crosslinked gelatins (23 kPa) (McDermott et al., “Mechanical Properties of Biomimetic Tissue Adhesives Based on the Microbial Transglutaminase-catalyzed Crosslinking of Gelatin”, Biomacromolecules, 5:1270-1279 (2004)), chitosans (3 kPa) (Ishihara et al., “Photo-crosslinkable Chitosan as a Dressing for Wound Occlusion and Accelerator in Healing Process”, Biomaterials, 23:833-840 (2002)), and fibrins (27 kPa) (Sierra et al., “A Method to Determine Shear Adhesive Strength of Fibrin Sealants”, Journal of Applied Biomaterials, 3:147-151 (1992)). The data cannot provide quantitative results for DERMABOND because cohesive failure occurred in all tests, meaning the actual adhesive strength may be higher than the observed failure strengths. Further tests of these adhesives can be conducted with stronger model tissues, such as crosslinked gelatins or skin.

### Example 3

Gelation kinetics. The rate of the NHS crosslinking reaction is known to vary strongly with pH (see U.S. Pat. No. 5,874,500 to Rhee et al., entitled “Crosslinked Polymer Compositions and Methods for Their Use”). Therefore, the effect of pH on the gelation time of liquid PEG-S/aECM mixtures was examined.

A series of buffers with varying pH were prepared by mixing separate solutions of 300 mM NaH₂PO₄ (sodium phosphate) and 300 mM Na₂CO₃ (sodium carbonate). These buffers were selected from procedures found in the literature and were referred to as “PCx buffers”, where x is the pH (see U.S. Pat. No. 6,312,725 to Wallace et al., entitled “Rapid Gelling Biocompatible Polymer Composition”). Fifty (50) μL of 15% (w/v) aECM solution and 50 μL of PCx buffer were added to a cylindrical glass vial with a 10 mm inner diameter. A square prism magnetic stir bar with a length of 8.0 mm and a width of 1.6 mm was placed in the vial and began stifling. Fifty (50) μL of 25% (w/v) unpurified PEG-S solution was added to the vial, 60.0 s (seconds) after the PEG-S solution was prepared. The liquid solution completely covered the stir bar. A video camera was used to observe and record the motion of the stir bar, which indicated the phase of the PEG-S/aECM mixture. In the method described herein, a stir bar both provided mechanical mixing of the PEG-S and aECM solutions and also allowed visual observation of the abrupt increase in viscosity upon gelation. The time between the introduction of PEG-S to aECM and the moment the stir bar stops its regular motion was taken to be the “gelation time”.

The results confirmed that the NHS crosslinking reaction was accelerated with increasing pH. FIG. 8 is an illustration of a graph 80 showing the results of the gelation time of PEG-S/aECM in seconds versus the pH of PCx buffer. The tests were conducted at each pH, 0.5 increments between 7 and 9.5. This rudimentary experiment provided evidence that gelation time decreased with increasing environment pH. One with skill in the art realizes that this knowledge can be used for fine tuning of the adhesive curing time, as some surgical applications might require instant curing while others might require more time for the clinician to position the adhered tissues.

### Example 4

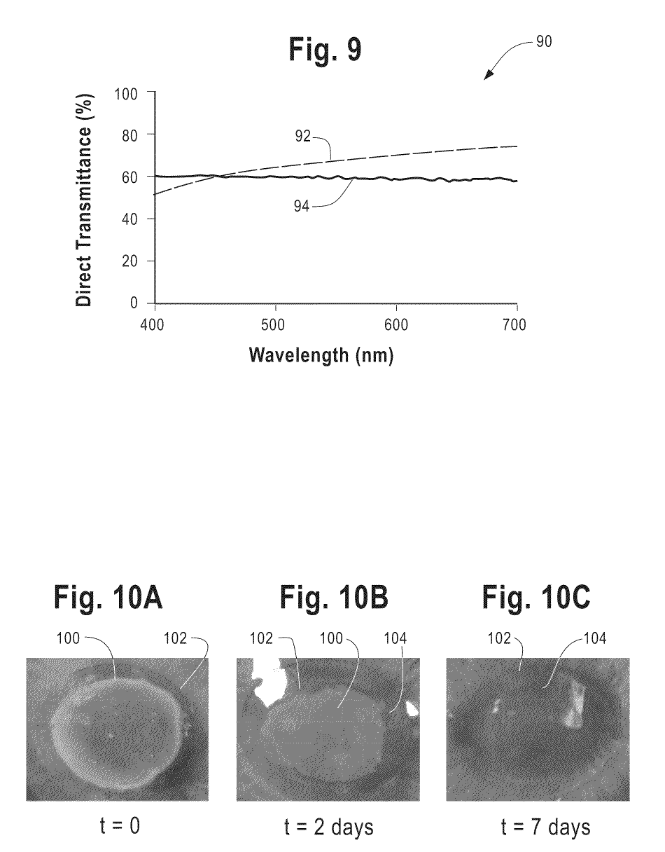

Gel transparency. The PEG-S/aECM formulation must be transparent in order to function as a viable tissue adhesive, such as a corneal adhesive. Visual examination of the PEG-S/aECM mixture suggested that the adhesive is sufficiently transparent for corneal applications. However, it is instructive to quantitatively examine optical properties during gelation. UV/Vis spectroscopy can be used to quickly assess the transparency of PEG-S/aECM mixtures before and after gelation. Equal volumes of 25% (w/v) purified PEG-S and 15% aECM solution were premixed, and 54, of the premix was analyzed using a NanoVue spectrophotometer (GE Healthcare of Piscataway, N.J.). The path length was 0.5 mm and deionized water was used as a blank. FIG. 9 is an illustration of a graph 90 showing the direct transmittance (%) versus wavelength (nm (nanometers)) of cured PEG-S/aECM adhesive compared to a human cornea. Plot line 92 indicates data from a human cornea obtained from the literature (Boettner et al., “Transmission of the Ocular Media”, Investigative Opthalmology and Visual Science, 1:776-783 (1962)). Plot line 94 indicates the results for PEG-S/aECM adhesive after ninety (90) minutes of curing. The results showed that the transmittance of a 0.5 mm thick layer of adhesive was similar to the total transmittance of the human cornea. Since the thickness of the human cornea is 0.5 mm-0.6 mm (see Boettner et al., “Transmission of the Ocular Media”, Investigative Opthalmology and Visual Science, 1:776-783 (1962)), the PEG-S/aECM has optical properties that are similar to the human cornea. One with skill in the art realizes that an actual adhesive layer should be much thinner than 0.5 mm, so embodiments disclosed herein have sufficient transparency for ophthalmic applications.

### Example 5

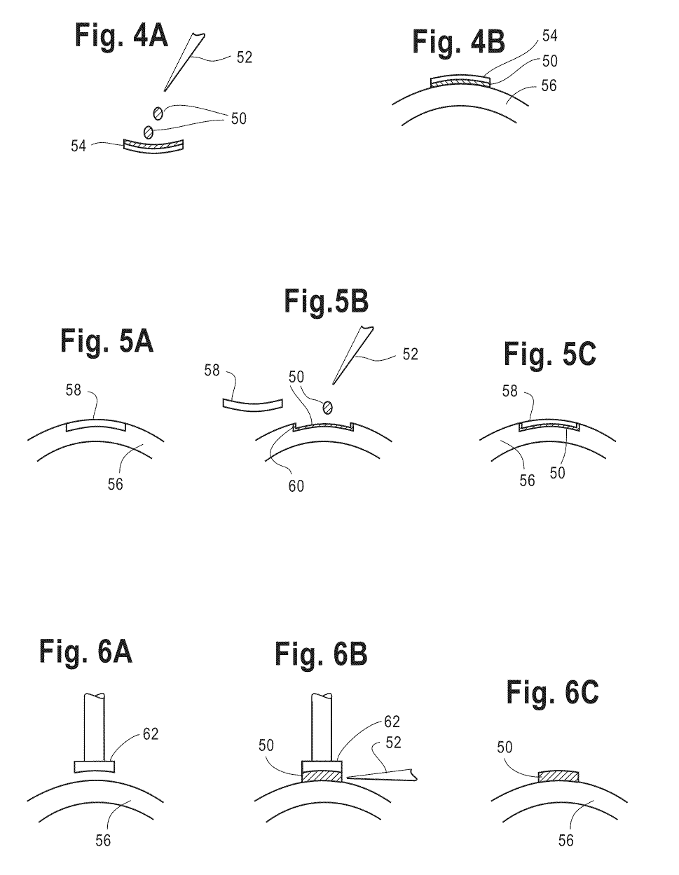

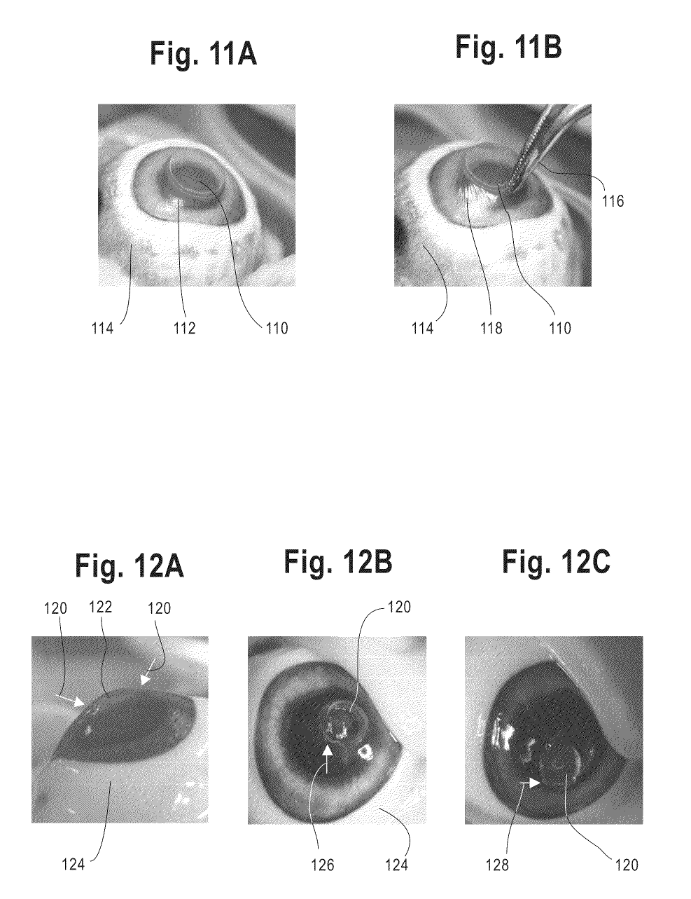

Mock Surgeries. Various mock surgeries were performed on porcine cadaver eyes in vitro, as shown in schematic illustrations FIGS. 4A-6C and clinical exam photographs FIGS. 11A-13C. Gelatin disks were applied to the stroma as an analogy to adhering a corneal onlay. These studies demonstrated possible uses of the PEG-S/aECM formulation and its favorable qualities. As mentioned previously, the corneal epithelium was debrided with a scalpel blade before all tests. All application surfaces were soaked with PC9.5 buffer for at least ten (10) minutes before application of the PEG-S/aECM formulation. The adhesive premix was composed of equal volumes of 25% (w/v) purified PEG-S and 15% aECM solution.

FIGS. 4A-6C show schematic illustrations of various mock surgeries performed on porcine cadaver eyes. In one mock surgery, as shown in FIGS. 4A-4B, the use of PEG-S/aECM for attaching corneal onlays was conducted. FIG. 4A is a schematic illustration of a mock surgery showing PEG-S/aECM adhesive 50 applied to the gelatin disk 54 via a dropper 52. Two (2) μL of droplets of PEG-S/aECM adhesive 50 was applied to the gelatin disk 54 having a diameter of 8 mm and a thickness of 1 mm. FIG. 4B is a schematic illustration of a mock surgery showing the gelatin disk 54 with the PEG-S/aECM adhesive 50 of FIG. 4A applied to the stroma 56 of an eye to form a corneal onlay. The gelatin disk 54 was immediately placed upon the stroma 56 and allowed to cure for fifteen (15) minutes.

In another mock surgery, a cut anterior portion of the stroma was removed by trephine and then reattached with PEG-S/aECM. FIG. 5A is a schematic illustration of a mock surgery showing a cut anterior portion 58 of the stroma 56 of an eye. A 4 mm diameter anterior portion hole was cut in the stroma 56 by trephine (not shown), and a scalpel (not shown) was slid under the cut to remove about half the corneal thickness. A trephine is a surgical instrument with a cylindrical blade. FIG. 5B is a schematic illustration of a mock surgery showing the cut anterior portion 58 of FIG. 5A removed and a droplet of PEG-S/aECM adhesive 50 applied to a cavity opening 60 in the stroma 56. One or more droplets of the PEG-S/aECM adhesive 50 were applied via dropper 52 in the cavity opening 60. FIG. 5C is a schematic illustration of a mock surgery showing the removed cut anterior portion 58 of FIG. 5B reattached to the stroma 56 with the PEG-S/aECM adhesive 50. The PEG-S/aECM adhesive was cured for fifteen (15) minutes after reattachment to the stroma.

In another mock surgery, a glass lens was used to mold an in situ forming corneal onlay, and PEG-S/aECM adhesive was molded by the glass lens into a corneal onlay directly onto a stroma. FIG. 6A is a schematic illustration of a mock surgery showing a glass lens 62 over the stroma 56 of an eye. A glass lens obtained from Rolyn Optics Co. of Covina, Calif., having an 8.0 mm diameter, −29.0 mm focal length, was coated with nail polish (SALLY HANSEN Teflon Tuff from Coty US LLC of New York, N.Y. —SALLY HANSEN is a registered trademark of Coty US LLC of New York, N.Y.) to enhance hydrophobicity and easy removal after curing. The glass lens was brought a distance of 0.5 mm away from the stroma. FIG. 6B is a schematic illustration of a mock surgery showing a PEGS/aECM adhesive 50 inserted between the glass lens 62 and the stroma 56 of the eye. Twenty-five (25) μL of PEG-S solution, 25 μL of aECM solution, and 10 μL of PC8.5 buffer were premixed, and 50 μL of this premix was pipetted into the gap between the glass lens and the stroma. After twenty (20) minutes of curing, the glass lens was removed. FIG. 6C is a schematic illustration of a mock surgery showing a molded in situ corneal onlay on the stroma of an eye where the PEG-S/aECM adhesive is in the form of the molded in situ corneal onlay 50.

FIGS. 11A-13C show clinical exam photographs of various mock surgeries performed on porcine cadaver eyes. In one mock surgery performed on porcine cadaver eyes, as shown in FIGS. 11A-11B, the use of PEG-S/aECM for attaching corneal onlays was conducted. FIG. 11A shows a clinical exam photograph of a mock surgery performed on a porcine cadaver eye depicting a gelatin disk 110 adhered to the stroma 114 of the porcine cadaver eye with PEG-S/aECM adhesive 112. FIG. 11B shows a clinical exam photograph of a mock surgery performed on a porcine cadaver eye depicting forceps 116 applying shear force 118 to the gelatin disk 110 of FIG. 11A. The gelatin disk 110 was applied to the stroma 114 as an analogy to adhering a corneal onlay. Remarkable optical clarity through the gelatin disk 110, adhesive 112 bond, and stroma 114 can be seen, and the iris and pupil were completely visible and unobscured. Mechanical strength of the adhesive bond is shown in FIG. 11B, where the corneal tissue deformed considerably as a result of the shear force 118 applied to the gelatin disk 110 onlay. It was expected that onlays attached with PEG-S/aECM adhesive would easily resist common disturbances from blinking and rubbing eyes.

In another mock surgery performed on porcine cadaver eyes, as shown in FIGS. 12A-12C, a cut anterior portion of the stroma was removed by trephine and then reattached with PEG-S/aECM FIG. FIG. 12A shows a clinical exam photograph of a mock surgery performed on a porcine cadaver eye depicting a side view of a reattached piece of stroma 120 of porcine cadaver eye 124 reattached with a PEG-S/aECM adhesive 122. The arrows in FIG. 12A indicate edges of the reattached piece of stroma 120. FIG. 12B shows a clinical exam photograph of a mock surgery performed on a porcine cadaver eye depicting a cavity opening in the stroma of the eye 124 where the reattached piece of stroma 120 of FIG. 12A was removed along with a piece of surrounding stroma 126. The arrow in FIG. 12B indicates a piece of the remaining tissue that is missing. FIG. 12C shows a clinical exam photograph of a mock surgery performed on a porcine cadaver eye depicting the removed reattached piece of stroma 120 with the piece of surrounding stroma 126 still attached via the PEG-S/aECM adhesive 128. The missing tissue is still attached to the removed piece by the adhesive. The arrow in FIG. 12C indicates the piece of the remaining tissue that is missing. The stromal reattachment surgeries (see also FIGS. 5A-5C) emulate anterior lamellar keratoplasties and reattachment of LASIK (laser-assisted in situ keratomileusis) flaps. In both cases a tissue section must be glued into a cavity in the stroma. Application of PEG-S/aECM resulted in seamless reattachment of the corneal tissue. In fact, it was difficult to find the boundaries of the reattached tissue even though they were marked in FIG. 12A. This provided further confirmation of the adhesive's favorable optical properties. The reattachment was mechanically successful as well. The eyelid could be swept over the repaired surface with no observed damage. Even rubbing the adhesive bond with forceps showed no motion of the reattached tissue. When forceps (not shown) were used to pierce the adhesive bond and pry the tissue away (FIG. 12B), a piece of the surrounding stroma was removed along with the previously trephined section. This piece was still attached to the section via the PEG-S/aECM adhesive. These results showed that in some cases, the adhesive bond was even stronger than the corneal tissue.

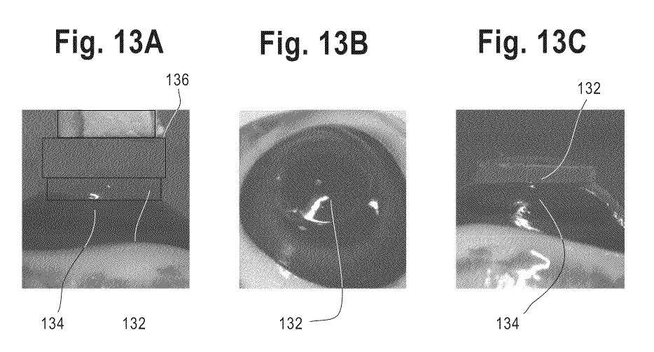

In another mock surgery performed on porcine cadaver eyes, as shown in FIGS. 13A-13C, a glass lens was used to mold an in situ forming corneal onlay, and PEG-S/aECM adhesive was molded by the glass lens into a corneal onlay directly onto a stroma. FIG. 13A shows a clinical exam photograph of a mock surgery performed on a porcine cadaver eye depicting a molded in situ corneal onlay 132 molded between a glass lens 136 and a stroma 134 of an eye. FIG. 13B shows a clinical exam photograph of a mock surgery performed on a porcine cadaver eye depicting a top view of the molded in situ corneal onlay 132 of FIG. 13A. FIG. 13C shows a clinical exam photograph of a mock surgery performed on a porcine cadaver eye depicting a side view of the molded in situ corneal onlay 132 over the stroma 134 of FIG. 13A. The molded in situ onlays are a possible refractive correction therapy, where PEG-S/aECM adhesive was used for its bulk optical properties rather than its mechanical adhesive strength. Rather than using a corneal adhesive to attach a corneal onlay (made out of aECM, for example), in this procedure the adhesive became the onlay itself. As a primitive prototype, a glass lens was used to mold an extremely thick gel on the anterior stromal surface. Removal of the lens gave a smooth gel surface which retained the curvature of the mold (FIG. 13C). However, small unidentified dots were observed in the bulk gel, and it was believed that these could be entrained air bubbles. Overall, the result was encouraging as it showed that PEG-S/aECM adhesive can be molded into a custom-shaped gel attached to the stromal surface of the eye. For further development of this technology, thinner onlays can be investigated as well as a mold that can be even further adaptable to the cornea. Additionally, it will be important to ascertain the swelling behavior of bulk PEG-S/aECM in the ocular environment, as this may change the refractive power of the in situ onlays.

### CONCLUSION

As shown in the above disclosed Examples, the potential of PEG-S/aECM as a tissue adhesive, such as a corneal adhesive, has been shown. Many of the stated criteria for the ideal tissue adhesive, such as a corneal adhesive, have been addressed. This formulation compared well against known adhesives in the following characteristics: (1) mechanical strength, and mock surgeries demonstrated superior performance in vitro. Visible spectroscopy gave preliminary data indicating good (2) transparency, and mock surgeries confirmed the result. Previous studies have shown sufficient (3) biocompatibility for both components independently, as PEG-S is used in FDA-approved adhesives and aECM has passed considerable scrutiny in ophthalmic applications. With regard to (4) re-epitheliazation, it has been demonstrated in known work on crosslinked aECM films.

The two component formulation of an engineered protein and a polymer crosslinker of the tissue adhesive of the disclosure was subjected to a series of experiments that established its potential as a superior tissue adhesive. A customized method was developed which compared embodiments of the tissue adhesive of the disclosure with known adhesives. Kinetics, optical, and transport properties of the adhesive were characterized. Ophthalmic surgeries were performed in vitro which demonstrated novel uses and favorable qualities of embodiments of the tissue adhesive of the disclosure. Overall, the embodiments of the tissue adhesive formulation of the disclosure demonstrated considerable mechanical strength, transparency, and biocompatibility. Additionally, compelling evidence was cited for its ability to regrow the corneal epithelium.

Further, it is known that when dissimilar polymers are crosslinked together, a hazy appearance typically results. However, with the combination of an engineered protein, such as aECM, and a polymer crosslinker, such as PEG-S, the protein-polymer hydrogel that was formed resulted in unexpected and surprising optical clarity through the corneal tissue, the adhesive bond, and the stroma of the eye. Further, it is known that incorporation of a synthetic polymer with a protein can impede cell adhesion and migration. However, with the combination of an engineered protein, such as aECM, and a polymer crosslinker, such as PEG-S, the protein-polymer hydrogel that was formed resulted in unexpected and surprisingly good cell adhesion and migration.

In summary, there is provided in one embodiment of the disclosure a tissue adhesive composition comprising an engineered protein having repeated blocks of an elastin domain and at least one cell-binding domain and further comprising a polymer crosslinker. When the engineered protein and the polymer crosslinker are introduced onto a tissue, the engineered protein and the polymer crosslinker initiate an in situ crosslinking reaction to form an adhesive bond that is mechanically strong, transparent, biocompatible, and stimulates regrowth of one or more tissue layers over the adhesive bond. In another embodiment of the disclosure there is provided a molded corneal onlay and method of making the same.

Many modifications and other embodiments of the disclosure will come to mind to one skilled in the art to which this disclosure pertains having the benefit of the teachings presented in the foregoing descriptions and the associated drawings. The embodiments described herein are meant to be illustrative and are not intended to be limiting. Although specific terms are employed herein, they are used in a generic and descriptive sense only and not for purposes of limitation.

What is claimed is:

1. A tissue adhesive composition comprising:
an engineered protein having repeated blocks of an elastin domain and at least one cell-binding domain; and,
a polymer crosslinker,
wherein when the engineered protein and the polymer crosslinker are introduced onto a tissue, the engineered protein and the polymer crosslinker initiate an in situ crosslinking reaction to form an adhesive bond that is mechanically strong, transparent, biocompatible, and stimulates regrowth of one or more tissue layers over the adhesive bond.

2. The tissue adhesive composition of claim 1 wherein the engineered protein is aECM-RGD comprising SEQ ID NO: 1.

3. The tissue adhesive composition of claim 1 wherein the elastin domain comprises one of SEQ ID NO: 2-4, 11, 12-19, or 20.

4. The tissue adhesive composition of claim 1 wherein the cell-binding domain comprises a fibronectin domain comprising one of SEQ ID NO: 26 or 27.

5. The tissue adhesive composition of claim 1 wherein the polymer crosslinker is selected from the group comprising a linear telechelic PEG (polyethylene glycol) and a star PEG (polyethylene glycol) with two or more arms.

6. The tissue adhesive composition of claim 5 wherein the polymer crosslinker comprises a four-arm polyethylene glycol with succinimidyl glutarate end groups (PEG-S).

7. The tissue adhesive composition of claim 1 wherein the tissue adhesive composition is molded into a corneal onlay in a mammalian eye.

8. The tissue adhesive composition of claim 1 wherein the tissue adhesive composition has applications selected from the group comprising sealing corneal ulcers and perforations, reducing or eliminating the need for sutures in keratoplasties, adhering corneal onlays onto a stroma for vision correction, reattaching LASIK (laser-assisted in situ keratomileusis) flaps, correcting refractive errors, and providing vascular tissue grafts.

9. The tissue adhesive composition of claim 1 wherein the engineered protein is present in an amount of from about 10% weight per volume (w/v) to about 40% weight per volume (w/v) based on the total weight per volume of the tissue adhesive composition, and wherein the polymer crosslinker is present in an amount of from about 10% weight per volume (w/v) to about 40% weight per volume (w/v) based on the total weight per volume of the tissue adhesive composition.

10. The tissue adhesive composition of claim 1 wherein the tissue adhesive composition comprises a corneal adhesive for use in a mammalian eye.

11. A molded corneal onlay for use in a mammalian eye, comprising:
a bulk hydrogel comprising an engineered protein having repeated blocks of an elastin domain and at least one cell-binding domain, and further comprising a polymer crosslinker,
wherein the bulk hydrogel is molded on a corneal surface to form a molded corneal onlay, and the engineered protein and the polymer crosslinker initiate an in situ crosslinking reaction to attach the molded corneal onlay to the corneal surface, and further wherein the molded corneal onlay is optically transparent, biocompatible, protects the corneal surface, is used to correct refractive errors, and stimulates cellular regrowth of corneal cells.

12. The molded corneal onlay of claim 11 wherein the engineered protein is aECM-RGD comprising SEQ ID NO: 1.

13. The molded corneal onlay of claim 11 wherein the polymer crosslinker is selected from the group comprising a linear telechelic PEG (polyethylene glycol) and a star PEG (polyethylene glycol) with two or more arms.

14. The molded corneal onlay of claim 13 wherein the polymer crosslinker comprises a four-arm polyethylene glycol with succinimidyl glutarate end groups (PEG-S).

15. A method of adhering tissue comprising:
applying a tissue adhesive composition to one or more tissue surfaces, the tissue adhesive composition comprising:
an engineered protein having repeated blocks of an elastin domain and at least one cell-binding domain; and
a polymer crosslinker,
wherein when the engineered protein and the polymer crosslinker are applied to the one or more tissue surfaces, the engineered protein and the polymer crosslinker initiate an in situ crosslinking reaction to form an adhesive bond; and,
curing the tissue adhesive composition to bond the composition to the one or more tissue surfaces and to provide a cured adhesive bond that is mechanically strong, transparent, biocompatible, and stimulates regrowth of one or more tissue layers over the cured adhesive bond.

16. The method of claim 15 wherein the engineered protein is aECM-RGD comprising SEQ ID NO: 1.

17. The method of claim 15 wherein the polymer crosslinker is selected from the group comprising a linear telechelic PEG (polyethylene glycol) and a star PEG (polyethylene glycol) with two or more arms.

18. A method of making a molded corneal onlay for use in a mammalian eye, comprising:
providing a bulk hydrogel comprising an engineered protein having repeated blocks of an elastin domain and at least one cell-binding domain, and further comprising a polymer crosslinker;
molding the bulk hydrogel on a corneal surface to form a molded corneal onlay; and,
attaching the molded corneal onlay to the corneal surface via the engineered protein and the polymer crosslinker initiating an in situ crosslinking reaction,
wherein the molded corneal onlay is optically transparent, biocompatible, protects the corneal surface, is used to correct refractive errors, and stimulates cellular regrowth of corneal cells.

19. The method of claim 18 wherein the engineered protein is aECM-RGD comprising SEQ ID NO: 1.

20. The method of claim 18 wherein the polymer crosslinker is selected from the group comprising a linear telechelic PEG (polyethylene glycol) and a star PEG (polyethylene glycol) with two or more arms.
